SEQUENCE LISTING (5 sequences)

SEQ ID NO: 1 - DNA, 21 bp, Artificial Sequence
  shRNA
gcaaatggacacaaattacaa

SEQ ID NO: 2 - DNA, 30 bp, Artificial Sequence
  primer
gcagctcaggaagaatcaaagctggctgcc

SEQ ID NO: 3 - DNA, 30 bp, Artificial Sequence
  primer
ggcagccagctttgattcttcctgagctgc

SEQ ID NO: 4 - DNA, 30 bp, Artificial Sequence
  primer
aagctggctgccaaatctgagcgagtgaga

SEQ ID NO: 5 - DNA, 30 bp, Artificial Sequence
  primer
tctcactcgctcagatttggcagccagctt

(19) United States
(12) Patent Application Publication
(10) Pub. No.: US 2022/0403044 A1
(43) Pub. Date: Dec. 22, 2022

(54) USE OF ANTI-EPCAM ANTIBODIES IN CANCER THERAPY

(71) Applicant: ACADEMIA SINICA, Taipei (TW)

(72) Inventors: Han-Chung WU, Taipei City (TW); Hao-Nien CHEN, Miaoli City (TW)

(21) Appl. No.: 17/756,022

(22) PCT Filed: Nov. 16, 2020
(86) PCT No.: PCT/US2020/060746

§ 371 Date: May 13, 2022

Related U.S. Application Data
(60) Provisional application No. 62/935,470, filed on Nov. 14, 2019.

(51) Int. Cl. C07K 16/30; A61P 35/00; A61P 11/00; C07K 16/28; A61P 15/00; A61P 1/00; C12N 15/113

(52) U.S. Cl. CPC C07K 16/30; A61P 35/00; A61P 11/00; C07K 16/2827; A61P 15/00; A61P 1/00; C12N 15/1138; C07K 2317/24; A61K 2039/505

(57) ABSTRACT

The present disclosure relates to therapeutic use of anti-EpCAM antibodies. Also provided is a combination immune therapy of an anti-EpCAM antibody and a PD-L1 immune checkpoint inhibitor.

### PRIORITY

This application is a 371 National Phase of International Patent Application No. PCT/US2020/060746, filed Nov. 16, 2020, which claims the benefit of priority under 35 U.S.C. 119(e) to U.S. Provisional Application No. 62/935,470, filed 14 Nov. 2019, which is incorporated herein by reference in its entirety.

### SEQUENCE LISTING

The instant application contains a Sequence Listing which has been submitted electronically in ASCII format and is hereby incorporated by reference in its entirety. Said ASCII copy, created on May 13, 2022, is named G4590-10700NP_SeqListing_20220513.txt and is 2 kilobytes in size.

### FIELD OF THE INVENTION

The present disclosure relates to therapeutic use of anti-EpCAM antibodies. Particularly, combination immune therapy of an anti-EpCAM antibody and a PD-L1 immune checkpoint inhibitor is described.

### BACKGROUND OF THE INVENTION

Epithelial cell adhesion molecule (EpCAM) is the most frequently expressed tumor-associated antigen; it is overexpressed in the majority of human adenocarcinomas and squamous cell carcinomas and is associated with poor prognosis in patients. EpCAM is sequentially processed by a disintegrin and metalloproteinase 17 (ADAM17), also called tumor necrosis factor alpha converting enzyme (TACE), and γ-secretase. Processing by these enzymes respectively releases the extracellular domain (EpEX) and intracellular domain (EpICD). After release, EpICD interacts with four and a half LEVI domains protein 2 (FHL2) and β-catenin to form a complex that translocates to the nucleus and interacts with Lef-1, which binds to DNA. The EpICD complex promotes tumorigenesis of tumor initiating cells (TICs) through the upregulation of reprogramming genes and epithelial-mesenchymal transition (EMT). Additionally, the increased release of EpEX enhances EpICD generation and upregulates the expression of reprogramming and EMT genes. It was reported that EpEX could directly bind to EGFR and stimulate EGFR phosphorylation and its downstream signaling pathway (5,8,10). Moreover, EpEX-induced EGFR phosphorylation can activate ADAM17 and γ-secretase to further increase the shedding of EpEX and EpICD (Liang K H, Tso H C, Hung S H, Kuan, I I, Lai J K, Ke F Y, et al. Extracellular domain of EpCAM enhances tumor progression through EGFR signaling in colon cancer 16 cells. Cancer Lett 2018; 433:165-75; Pan M, Schinke H, Luxenburger E, Kranz G, Shakhtour J, Libl D, et al. EpCAM ectodomain EpEX is a ligand of EGFR that counteracts EGF-mediated epithelial-mesenchymal transition through modulation of phospho-ERK1/2 in head and neck cancers. PLoS Biol 2018; 16:e2006624; and Liang K-H, Lai J-K, Kuan, II, Tso H-C, Wu H-C. EpCAM/EpEX regulate tumor progression through EGFR signaling in colon cancer cells. AACR; 2017). However, the domain of EpEX that binds and activates EGFR has not been previously identified.

### SUMMARY OF THE INVENTION

The present disclosure surprisingly found that EGF-like domain I within the extracellular domain of EpCAM (EpEX) binds EGFR, activating both AKT and MAPK signaling to inhibit FOXO3a function and stabilize PD-L1 protein, respectively. Treatment with the EpCAM-neutralizing antibody inhibits AKT and FOXO3a phosphorylation, increases FOXO3a nuclear translocation, and upregulates HtrA2 expression to promote apoptosis while decreasing PD-L1 protein levels to enhance the cytotoxic activity of CD8+ T cells. The findings in the present disclosure not only shed light on the molecular mechanisms underlying EpCAM signaling in cancer malignancy but also suggest therapeutic targeting of EpCAM may work well in combination with current immunotherapies. The combination of an anti-EpCAM antibody with an anti-PD-L1 antibody exhibits unexpected effect in tumor elimination and extension of survival of a subject in metastasis, suggesting a new combination therapy for cancer immunotherapy in subjects.

In one aspect, the present disclosure provides a method for treating, inhibiting or eliminating a cancer in a subject, comprising administering an effective amount of an inhibitor or an antagonist targeting to EGF-like domain I within the EpEX to the subject.

In one embodiment, the EGF-like domain I within the EpEX comprises a peptide consisting of amino acids 27 to 59 of EGF-like domain or a variant thereof that can bind to EGFR.

In some embodiments, the inhibitor or the antagonist targeting to EGF-like domain I within the EpEX is ribozymes, antisense oligonucleotides, short hairpin RNA (shRNA) molecules or small interfering RNA (siRNA) molecules that specifically inhibit and/or reduce the expression or activity of EpCAM. In some further embodiments, the inhibitor or the antagonist targeting to EGF-like domain I within the EpEX is a shRNA or a siRNA knockdown expression of EGFR, AKT, PD-L1 and/or MAPK and/or phosphorylation of FOXO3a and/or increases expression of HtrA2 and/or FOXO3a nuclear translocation. In a further embodiment, the shRNA comprises a nucleotide sequence consisting of GCAAATGGACACAAATTACAA (SEQ ID NO: 1) or a variant specifically inhibit and/or reduce the expression or activity of EpCAM.

In some other embodiments, the inhibitor or the antagonist targeting to EGF-like domain I within the EpEX is a small molecule, peptide, an antibody or antibody fragment that can partially or completely block EpCAM activity.

In some embodiments, the inhibitor or the antagonist targeting to EGF-like domain I within the EpEX is an EpCAM-neutralizing antibody. Preferably, the antibody is EpAb2-6 or a variant that can neutralize EpCAM.

In another aspect, the present disclosure provides a method for treating, inhibiting or eliminating a cancer in a subject, comprising administering to a subject in need thereof, an effective amount of an inhibitor or an antagonist targeting to EGF-like domain I within the EpEX, and an inhibitor or an antagonist targeting to PD-L1 in an amount effective to inhibit expression or activation of PD-L1.

In one embodiment, the inhibitor or the antagonist targeting to PD-L1 is a PD-L1 checkpoint inhibitor. In some embodiments, the PD-L1 checkpoint inhibitor is MEDI4736, atezolizumab, avelumab, or durvalumab.

In some embodiments, the inhibitor or the antagonist targeting to EGF-like domain I within the EpEX is intermittently, concurrently, separately or sequentially administered with the inhibitor or an antagonist targeting to PD-L1.

In some embodiments, the cancer is melanoma, renal cancer, prostate cancer, breast cancer, colorectal cancer, lung cancer, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer or T-cell lymphoma.

In one embodiment, the cancer is an EpCAM-overexpressing cancer, EGFR-overexpressing or activating cancer, AKT-overexpressing or overactivating cancer, MAPK-overexpressing or activating cancer, FOXO3a-inactivating cancer, HtrA2-inactivating cancer or PD-L1 expressing cancer.

In a further embodiment, the cancer is a metastatic cancer or an advanced cancer. In a further embodiment, the cancer is a metastatic colorectal cancer or small cell lung cancer or an advanced colorectal cancer or small cell lung cancer.

In one embodiment, the subject is EpCAM-overexpressing. In one embodiment, the subject who has been treated with at least one anti-cancer therapy or anti-cancer agent.

### BRIEF DESCRIPTION OF THE DRAWINGS

FIGS. 1A to 1H. EpEX binds to EGFR through its EGF-like domain I. (FIG. 1A) Immunoprecipitation (IP) of affinity cross-linked EpEX-Fc bound to EGFR from HCT116 cells. (FIG. 1B) EpEX-Fc was added to EGFR_{ECD}-coated ELISA plates and detected by TMB colorimetric peroxidase assay. (FIG. 1C) 293T cells were transfected with EGFR-Flag and EpCAM-V5. IP was performed with anti-V5 antibody (left panel) or anti-Flag antibody (right panel) followed by Western blotting. (FIG. 1D) 293T cells were transfected with EGFR-Flag and EGF domain-deleted mutant EpCAM-V5. The protein interaction was probed by IP with anti-V5 antibody and Western blotting with anti-Flag antibody. (FIG. 1E) In culture media from 293T cells with EGFR_{ECD}-Flag and EpEX-Fc transfection, IP was performed with Dynabeads® Protein G and detected by Western blotting with anti-Flag antibody. (FIG. 1F) 293T cells were transiently transfected with EGFR_{ECD}-Flag and EGF domain-deleted mutant EpEX-Fc. The EGFR_{ECD}-Flag and mutant EpEX-Fc interaction was examined by IP with Dynabeads® Protein G and Western blotting with anti-Flag antibody. (FIG. 1G) Purified EGFR_{ECD}-6×His and EGF domain-deleted mutant EpEX-Fc were incubated and IP was performed with Dynabeads® Protein G and detected by Western blotting. (FIG. 1H) HCT116 cells were starved and treated with wild-type or EGF domain-deleted mutant EpEX, and EGFR signaling was analyzed by Western blotting. N=3 independent experiments. Graphical data are shown as mean±SEM. ***p<0.001, two-tailed Student's t-test.

FIGS. 2A to 2H. EpEX prevents cell apoptosis via inhibition of FOXO3a in colon cancer cells. (FIG. 2A) HCT116 cells were starved and then treated with 2.5 μg/ml EpEX-Fc for the indicated times. Total cell lysate was examined by Western blotting. (FIG. 2B) After treating with AG1478 (5 μM) or Wortmannin (1 μM) for 1 h, cells were exposed to EpEX (2.5 μg/ml) for 1 h. The phosphorylation of FOXO3a was examined by Western blotting. (FIG. 2C) The level of FOXO3a in nuclear fractions of EpEX-treated cells was probed, upper panel. Serum-starved HCT116 cells were treated with EpEX-Fc for 2 h and the location of FOXO3a was assessed by immunofluorescence, lower panel. (FIG. 2D) HCT116 cells were starved, pre-treated with 20 μg/ml EpAb2-6, and then treated with EpEX for 15 min. The phosphorylation of AKT and FOXO3a was analyzed by Western blotting. (FIG. 2E) HCT116 cells were treated with 20 μg/ml EpAb2-6 for 6 h, and β-catenin and FOXO3a were detected in the nuclear fraction of HCT116 cells. (FIG. 2F) qPCR analysis of FOXO3a-related gene expression (BIM, p21 and FasL) in control IgG- or EpAb2-6-treated HCT116 cells. (FIG. 2G) HCT116 cell line were treated with EpAb2-6 (in vitro) and (FIG. 2H) mouse bearing HCT116 subcutaneous xenograft were treated with EpAb2-6 (in vivo). Arrows indicate cellular localization of FOXO3a in the nucleus. Quantification of the number of cells with nuclear FOXO3a and β-catenin is shown on treated with EpAb2-6 in vitro (FIG. 2G) and in vivo (FIG. 2H) (mean±SD, N=200-300 cells in each group, *p<0.05). Bar graphs show mean±SEM. *p<0.05, **p<0.01.

FIGS. 3A to 3H: EpEX prevents apoptosis by inhibiting FOXO3a-mediated HtrA2 expression. (FIG. 3A) HCT116 cells were treated with control IgG or EpAb2-6 for 6 h, and apoptosis-related proteins were probed by the human apoptosis array kit. (FIG. 3B) Expression of HtrA2 was examined by immunoblotting and qPCR analysis. (FIG. 3C) EpAb2-6 induced the release of mitochondrial apoptogenic proteins, HtrA2 and cytochrome c, in colon cancer cells. (FIG. 3D) The percentage of cells with mitochondrial membrane potential (Δψm) depolarization was measured after EpAb2-6 treatment. (FIG. 3E) HCT116 cells with transfection of human HtrA2 proximal promoter-driven luciferase reporter were treated with control IgG or EpAb2-6. Luciferase activity assays were performed to evaluate the activity of the HtrA2 promoter. Results are expressed as fold induction compared to the IgG control. Statistical differences were determined by One-way ANOVA and Bonferroni multiple comparison test. (FIG. 3F) Quantitative ChIP analysis of FOXO3a binding to HtrA2 promoters was performed. (FIG. 3G) HtrA2-knockdown HCT116 cells were treated with EpAb2-6, and the apoptotic cells were quantified by fluorescein annexin V-FITC/PI double labeling. Statistical differences were determined by One-way ANOVA and Bonferroni multiple comparison test. (FIG. 3H) HtrA2-knockdown HCT116 cells were treated with EpAb2-6, and the percentage of cells with mitochondrial membrane potential (Δψm) depolarization was measured. Statistical differences were determined by One-way ANOVA and Bonferroni multiple comparison test. N=3 independent experiments. Graphical data are shown as 11 mean±SEM. *p<0.05, **p<0.01, ***p<0.001.

FIGS. 4A to 4L: EpCAM is correlated with PD-L1 expression. (FIG. 4A) The tumor growth of shLuc or shEpCAM H441 cells in NSG mice with or without PBMC injection. (FIG. 4B) Relative tumor size was evaluated, and weights are shown in the graph. (FIG. 4C) Evaluation of CD8⁺ T cells in tumor tissue. (FIG. 4D) Western blotting and (FIG. 4E) qRT-PCR analysis of PD-L1 in H460 and H441 cells. (FIG. 4F) Flow cytometry analysis of PD-L1 and EpCAM in H460 and H441 cells. (FIG. 4G) H460 and H441 cells were treated with 50 μM cycloheximide (CHX) for the indicated intervals and analyzed by Western blotting. Protein expression over time is shown in the graph. (FIG. 4H) The protein and mRNA levels of PD-L1 in EpCAM-knockdown H441 cells were analyzed by Western blotting and qRT-PCR, respectively. (FIG. 4I) EpCAM-knockdown H441 cells were treated with 50 μM CHX for the indicated intervals, and PD-L1 expression was analyzed by Western blotting. Protein expression over time is shown in the graph. (FIG. 4J) The expression of exogenous PD-L1 in EpCAM-knockdown H441 cells was analyzed by Western blotting. (FIG. 4K) Immunohistochemical staining for EpCAM and PD-L1 was performed in the lung cancer tissue array, and the positive correlation between EpCAM and PD-L1 expression in lung cancer patients is shown. (FIG. 4L) GSEA enrichment profile of T cells activation and proliferation in the lung cancer specimen.

FIGS. 5A to 5J. EpEX stabilizes PD-L1 protein via EGFR-MEK signaling. (FIG. 5A) H441 cells were treated with TAPI (ADAM17 inhibitor) or DAPT (γ-secretase inhibitor) for 24 h, and PD-L1 expression was analyzed by Western blotting (left) and qRT-PCR (right). (FIG. 5B) H441 cells were treated with the indicated concentrations of EpEX or EGF for 1 h and the PD-L1 expression was analyzed by Western blotting. (FIG. 5C) After treated with EpEX (45 nM) or EGF (16.5 nM) for the indicated intervals, PD-L1 expression was analyzed by Western blotting (left) or qRT-PCR (right). (FIG. 5D) EGFR-knockdown H441 cells were treated with EpEX or EGF for 1 h, and PD-L1 expression was analyzed by Western blotting. (FIG. 5E) PD-L1 was determined by Western blotting in H441 cells pretreated with Gefitinib, U1026, or wortmannin for 1 h, followed by treatment with EpEX or EGF for 1 h. (FIG. 5F) H441 cells were treated with EpAb2-6 or isotype for 16 h, and PD-L1 expression was analyzed by Western blotting, upper panel. PD-L1 expression was analyzed in EpAb2-6- or isotype-treated H441-derived xenograft tumors, lower panel. (FIG. 5G) After incubating cells with EpAb2-6 for 16 h, the protein half-life of PD-L1 was measured by treatment with 50 μM CHX for the indicated intervals and Western blotting analysis. Protein expression over time is shown in the graph. (FIG. 5H) H441 cells expressing PD-L1-eGFP-Flag were treated with EpAb2-6 for 16 h and MG132 for 5 h. The polyubiquitination of PD-L1-eGFP-Flag was analyzed by Western blotting after immunoprecipitation with anti-Flag. (FIG. 5I) MCF7, BT474 and Ca127 cells were treated with EpAb2-6 or isotype for 16 h, and PD-L1 expression was analyzed by Western blotting. (FIG. 5J) After treatment with EpAb2-6 or isotype for 16 h, H441 cells were co-cultured with T cells for 16 h and the apoptotic cells were quantified by fluorescein Annexin V-FITC/DAPI double staining.

FIGS. 6A to 6H. EpAb2-6 prolongs median survival in metastasis and orthotopic CRC models and improves the efficacy of anti-PD-L1 therapy in a PBMC-CDX xenograft model. (FIG. 6A) HCT116 cells transiently expressing GFP were injected into the tail veins of SCID mice with EpAb2-6. Mice in the control group (without EpAb2-6 treatment) were injected with the same dose of control IgG. N=3 independent experiments. Data are shown as mean±SD. **p<0.01. (FIG. 6B) NOD/SCID mice were intravenously injected with 1×10⁶ SW620 cells, followed by treatment with either control IgG or EpAb2-6 (N=6). According to the survival curves, mice treated with EpAb2-6 exhibited a greater survival rate than those treated with control IgG. (FIG. 6C) NSG mice were orthotopically implanted with HCT116-Luc cells and then treated with control IgG or EpAb2-6 starting at 8 days after tumor inoculation. Antibody injections were performed every 2 days for 16 days, via the tail vein. Tumor growth was monitored by examining bioluminescence with the IVIS 200 Imaging System. (FIG. 6D) Kaplan-Meier survival plots and median survival are shown for both treatment groups (N=8 per group) (P<0.005, log-rank test). (FIG. 6E) A schema for the experiment to evaluate treatment efficacy of EpAb2-6 and/or Atezolizumab in the PBMC-cell line-derived xenograft (CDX) model. (FIG. 6F) The tumor growth of H441 cells in EpAb2-6 and/or Atezolizumab-treated PBMC-CDX mice. (FIG. 6G) Tumor weight was evaluated and shown in the graph. (FIG. 6H) CD8⁺ T cells were quantified in tumor tissue.

FIG. 7. EpCAM signaling regulates tumor progression.

FIGS. 8A to 8C: EGFR-AKT signaling pathway decreases nuclear localization of FOXO3a. (FIGS. 8A and 8B) HCT116 cells were serum starved then pre-treated with or without 10 μM MG132 for 1 h before being treated with EGF (100 ng/ml) and extracted at the indicated times. Nuclear and cytoplasmic fractions were analyzed by immunoblotting with the indicated antibodies. (FIG. 8C) HCT116 cells were treated with EGF for 2 h, and the subcellular localization of FOXO3a was assessed by immunofluorescence staining.

FIGS. 9A to 9G. EpAb2-6 decreases EpICD shedding and β-catenin nuclear translocation. HCT116 cells were treated with either control IgG (20 μg/ml) or EpAb2-6 (20 μg/ml) for 24 h. Cell proteins were subsequently examined using (FIG. 9A) γ-secretase and (FIG. 9B) TACE activity assay kits. (FIG. 9C) HCT116 cells were treated with EpAb2-6 (0-20 μg/ml) for 24 h, and the culture supernatants were immunoblotted with anti-EpEX antibody. (FIG. 9D) HCT116 cells were treated with EpAb2-6 (20 μg/ml) for 6 h, and the subcellular localization of active β-catenin was assessed by immunofluorescence staining. (FIG. 9E) Real-time qPCR analysis mRNA levels of reprogramming and EMT genes in EpAb2-6 treated HCT116 cells. (FIG. 9F) The effect of EpAb2-6 on colon cancer cells was examined by the anchorage-independence assay. Results from one representative field in each group are shown. Quantification of results of colony formation data is presented. (FIG. 9G) The effect of EpAb2-6 on colon cancer cells was examined by spheroid formation. Spheroid formation from colon cancer cells was counted in triplicate. Representative images of tumorspheres after treatment with IgG or EpAb2-6 are shown. Bar, 5 mm. N=3 independent experiments. Error bars indicate ±SD. *p<0.05, **p<0.01.

FIGS. 10A to 10D. EGFR, AKT or FOXO3a knockdown and EpCAM knockout decrease EpAb2-6-induced apoptosis. (FIG. 10A) EpCAM-knockout (KO), (FIG. 10B) EGFR-, (FIG. 10C) AKT-, (FIG. 10D) FOXO3 a-knockdown HCT116 cells were treated with EpAb2-6, and the apoptotic cells were quantified by fluorescein-conjugated annexin V-FITC/PI double labeling. N=3 independent experiments. Bar graphs show mean±SEM. *p<0.05, **p<0.01.

FIG. 11A to 11C. EpCAM expression stabilizes PD-L1 protein in H460 cells. After transfection with vehicle or EpCAM expression vector, the PD-L1 protein and mRNA expression levels were analyzed by (FIG. 11A) Western blotting or (FIG. 11B) qRT-PCR. (FIG. 11C) H460 cells were treated with 50 μM CHX for the indicated intervals, and PD-L1 expression was analyzed by Western blotting. Protein expression over time is shown in the graph.

FIGS. 12A to 12B. Expression of EpCAM and PD-L1 in a colon tumor tissue array. Immunohistochemical staining for (FIG. 12A) EpCAM and (FIG. 12B) PD-L1 was performed on a colon tumor tissue array.

FIGS. 13A to 13D. EpCAM is crucial for PD-L1 protein expression. (FIGS. 13A, B) After starvation and treatment with IFNγ for 16 h, H441 cells were treated with EpEX or EGF for 1 h and the PD-L1 expression was analyzed by (FIG. 13A) qRT-PCR and (FIG. 13B) Western blotting. (FIGS. 13C, D) H441 were treated with or without TAPI, DAPT and IFNγ for 24 h, and PD-L1 expression was analyzed by (FIG. 13C) qRT-PCR and (FIG. 13D) Western blotting.

FIGS. 14A to 14F. MAPK pathway increases PD-L1 protein stability. (FIG. 14A) H441 cells were treated with DMSO, U1026 or wortmannin for the indicated intervals, and PD-L1 expression was analyzed by Western blotting. (FIG. 14B) H441 cells were treated with U0126 for 6 h, and CHX for the indicated intervals, and PD-L1 expression was analyzed by Western blotting. (FIG. 14C) H441 cells expressing PD-L1-Myc were treated with trametinib, U0126 or SCH772984 for 6 h, and total cell lysate was subjected to Western blotting. (FIG. 14D) H441 cells expressing PD-L1-eGFP-Flag were treated with MG132 for 5 h. Total cell lysate was immunoprecipitated with anti-Flag antibody and the polyubiquitin of PD-L1-eGFP-Flag analyzed by Western blotting. (FIG. 14E) PD-L1 expression was analyzed in shLuc- or shGSK3β-expressing cells by Western blotting. (FIG. 14F) shLuc or shGSK3β cells were starved for 16 h and then treated with EpEX or EGF for 1 h. The total cell lysate was subjected to Western blotting.

FIG. 15. EpAb2-6 treatment shows no significant body weight change in the orthotopic model with HCT116-Luc tumors. Average body weight of each group is shown on the indicated days. Error bars show mean±SD.

### DETAILED DESCRIPTION OF THE INVENTION

### Definitions

In order for the present disclosure to be more readily understood, certain terms are first defined below. Additional definitions for the following terms and other terms may be set forth through the specification. If a definition of a term set forth below is inconsistent with a definition in an application or patent that is incorporated by reference, the definition set forth in this application should be used to understand the meaning of the term.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below.

As used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

As used herein, the terms “inhibiting,” “eliminating,” “decreasing,” “reducing” or “preventing,” or any variation of these terms, referred to herein, includes any measurable decrease or complete inhibition to achieve a desired result.

As used herein, the term “antibody” refers to an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also fragments thereof (such as Fab, Fab′, F(ab′)2, Fv), single chain (ScFv), mutants thereof, fusion proteins comprising an antibody portion (such as domain antibodies), and any other modified configuration of the immunoglobulin molecule that comprises an antigen recognition site. An antibody includes an antibody of any class, such as IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

As used herein, the term “individual” or “subject” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

As used herein, the term “treating” or “treatment” (and grammatical variations thereof such as “treat”) refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state and remission or improved prognosis.

As used herein, the term “effective amount” (or “therapeutically effective amount”) is an amount sufficient to affect a beneficial or desired clinical result upon treatment. In certain embodiments, a therapeutically effective amount refers to an amount that is able to achieve one or more of an anti-cancer effect, prolongation of survival and/or prolongation of period until relapse. For example, and not by way of limitation, a therapeutically effective amount can be an amount of an inhibitor or antagonist that that minimizes, prevents, reduces and/or alleviates the symptoms of a cancer.

As used herein, the term “anti-cancer effect” refers to one or more of a reduction in aggregate cancer cell mass, a reduction in cancer cell growth rate, a reduction in cancer progression, a reduction in cancer cell proliferation, a reduction in tumor mass, a reduction in tumor volume, a reduction in tumor cell proliferation, a reduction in tumor growth rate and/or a reduction in tumor metastasis. In certain embodiments, an anti-cancer effect can refer to a complete response, a partial response, a stable disease (without progression or relapse), a response with a later relapse or progression-free survival in a patient diagnosed with cancer.

As used herein, the term “metastatic cancer” refers to a cancer that has spread from the part of the body where it started (the primary site) to other parts of the body. When cancer cells break away from a tumor, they can travel to other parts of the body through the bloodstream or the lymph system.

As used herein, the term “advanced cancer” is most often used to describe cancers that cannot be cured. This means cancers that would not totally go away and stay away completely with treatment.

As used herein, the term “synergistic” refers to a combination of therapies which is more effective than the additive effects of the single therapies.

Cross-Talk Between EGF-Like Domain I within the EpEX, EGFR, AKT, FOXO3 and HtrA2

EpCAM-overexpressing carcinoma cells possess stem-cell-like features, and their presence results in high rates of recurrence, metastasis and drug resistance. AKT is a primary mediator of EGFR signaling that promotes cell survival partially by inactivating pro-apoptotic proteins. One such pro-apoptotic protein that is inactivated by AKT phosphorylation is Forkhead transcription factor O3a (FOXO3a). FOXO3a is also referred to as FKHRL-1 and is a member of the forkhead transcription factor family. Because genes activated by FOXO proteins generally function to limit cell growth and promote death, this family is thought of as tumor suppressors. When activated, FOXO3a accumulates in the nucleus, where it enhances the transcription of various genes involved in apoptosis and the cell cycle control, such as BIM, FasL and p21. Furthermore, AKT inactivation is an essential step in anoikis, and FOXO3a regulation by the PI3K/AKT pathway may be essential for this inactivation.

The present disclosure reports that the EGF-like domain I within EpEX binds to EGFR and activates both the AKT and ERK1/2 pathways. EpEX-induced AKT activation inhibits FOXO3a activity, which decreases HtrA2 transcription. Moreover, an inhibitor or an antagonist targeting to EGF-like domain I within the EpEX (such as a therapeutic antibody directed against EpCAM (preferably EpAb2-6)) promotes FOXO3a nuclear translocation and increases HtrA2 expression. Accordingly, the present disclosure provides a method for treating, inhibiting or eliminating a cancer in a subject, comprising administering an effective amount of an inhibitor or an antagonist targeting to EGF-like domain I within the EpEX to the subject.

Those skilled in the art will be able to determine appropriate dosage amounts for the inhibitor or antagonist targeting to EGF-like domain I within the EpEX. The exact amount required will vary from subject to subject, depending on the disease state, physical conditions, age, sex, species and weight of the subject, the specific identity and formulation of the composition, etc. Dosage regimens may be adjusted to induce the optimum therapeutic response. For example, several divided doses may be administered daily or the dose may be proportionally reduced as indicated by the exigencies of the therapeutic situation. An appropriate effective amount can be determined by one of ordinary skill in the art using only routine experimentation.

Inhibitors or Antagonists Targeting to EGF-Like Domain I

In EpCAM extracellular domain, it contains two EGF-like domains at amino acids 27-59 (1^{st} EGF-like domain) and 66-135 (2^{nd} EGF-like domain), and a cysteine-free motif (Schnell U, Kuipers J, Giepmans B N. EpCAM proteolysis: new fragments with distinct functions? Bioscience reports 2013; 33:e00030). Surprisingly, the EGF-like domain I-deleted EpCAM mutant (EpCAM_{ΔEGFI}) shows decreased binding to EGFR, while the EGF-like domain II-deleted EpCAM mutant (EpCAM_{ΔEGFII}) exhibited an enhanced interaction with EGFR. In certain embodiments of the present disclosure, the EGF-like domain I within the EpEX comprises a peptide consisting of amino acids 27 to 59 of EGF-like domain or a variant thereof that can bind to EGFR.

The inhibitors or antagonists of the EGF-like domain I include compounds, molecules, chemicals, polypeptides and proteins that target to EGF-like domain I and they can activate both the AKT and ERK1/2 pathways. The present disclosure also unexpected found that the inhibitors or antagonists of the EGF-like domain I can suppress cancer malignancy through inhibition of tumorsphere formation. Furthermore, treatment of the inhibitor or antagonist targeting to EGF-like domain I within the EpEX markedly prolongs the survival of a subject in metastasis and in cancer.

Non-limiting examples of inhibitors or antagonists of the EGF-like domain I include ribozymes, antisense oligonucleotides, short hairpin RNA (shRNA) molecules and a small interfering RNA (siRNA) molecules that specifically inhibit and/or reduce the expression or activity of EpCAM. The inhibitors or antagonists of the EGF-like domain I knockdown expression of EGFR, AKT, PD-L1 and/or MAPK and/or phosphorylation of FOXO3a and/or increases expression of HtrA2 and/or FOXO3a nuclear translocation. In some examples, inhibitor or antagonist of the EGF-like domain I is an antisense, shRNA or siRNA nucleic acid sequence homologous to at least a portion of a EGF-like domain I nucleic acid sequence, which inhibits and/or reduces the expression or activity of EpCAM and knockdowns expression of EGFR, AKT, PD-L1 and/or MAPK and/or phosphorylation of FOXO3a and/or increases expression of HtrA2 and/or FOXO3a nuclear translocation. The homology of the portion relative to the EGF-like domain I sequence is at least about 75 or at least about 80 or at least about 85 or at least about 90 or at least about 95 or at least about 98 percent, where percent homology can be determined by, for example, BLAST or FASTA software. In certain non-limiting embodiments, the complementary portion can constitute at least 10 nucleotides or at least 15 nucleotides or at least 20 nucleotides or at least 25 nucleotides or at least 30 nucleotides and the antisense nucleic acid, shRNA or siRNA molecules can be up to 15 or up to 20 or up to 25 or up to 30 or up to 35 or up to 40 or up to 45 or up to 50 or up to 75 or up to 100 nucleotides in length. Antisense, shRNA or siRNA molecules can include DNA or atypical or non-naturally occurring residues, for example, but not limited to, phosphorothioate residues. Preferably, the shRNA comprises a nucleotide sequence consisting of GCAAATGGACACAAATTACAA (SEQ ID NO: 1) or a variant specifically inhibit and/or reduce the expression or activity of EpCAM.

The RNA molecules of the present disclosure can be expressed from a vector or produced chemically or synthetically. Methods for selecting an appropriate dsRNA or dsRNA-encoding vector are well known in the art for genes whose sequence is known.

In certain non-limiting embodiments, the inhibitors or antagonists of the EGF-like domain I can be a small molecule, peptide, an antibody or antibody fragment that can partially or completely block EpCAM activity. The inhibitor or an antagonist targeting to the EGF-like domain I within the EpEX can be an EpCAM-neutralizing antibody targeting the domain I. Preferably, the antibody is EpAb2-6 (UniProt ID: P16422) or a variant that can targets the domain I and neutralize EpCAM.

EpAb2-6 targets EpEX to block it and induces apoptosis (Liang K H, Tso H C, Hung S H, Kuan, I I, Lai J K, Ke F Y, et al. Extracellular domain of EpCAM enhances tumor progression through EGFR signaling in colon cancer 16 cells. Cancer Lett 2018; 433:165-75; Liao M Y, Lai J K, Kuo M Y, Lu R M, Lin C W, Cheng P C, et al. An anti-EpCAM antibody EpAb2-6 for the treatment of colon cancer. Oncotarget 2015; 6:24947-68). By blocking the function of EpEX, EpAb2-6 treatment interrupts the EpEX/EGFR/ADAM17 axis, which represents a positive feedback loop promoting EpCAM cleavage and subsequently increases EpEX and EpICD production. The present disclosure surprisingly found that EpAb2-6 treatment inhibits tumorsphere formation and thus EpAb2-6 is able to suppress cancer malignancy.

Combination of an Inhibitor or an Antagonist Targeting to EGF-Like Domain I within the EpEX, and an Inhibitor or an Antagonist Targeting to PD-L1

In addition to its mediation of survival signals, EGFR activation is crucial for triggering immune escape. EGFR-activating mutations were reported to be associated with increased PD-L1 expression in mouse models of EGFR-driven lung cancer and bronchial epithelial cells with the expression of mutant EGFR. Moreover, EGFR inhibitors can reduce PD-L1 expression in NSCLC cell lines with activated EGFR, suggesting that EGFR signaling may trigger immune escape. Further investigations demonstrated that EGFR ligands, such as EGF, induce PD-L1 expression primarily at the level of post-translational modifications. EGF was shown to stabilize PD-L1 by inducing PD-L1 glycosylation, which prevents GSK3-dependent proteasomal degradation of PD-L1 by β-TrCP.

Moreover, the MAPK signaling pathway, another important axis of EGFR signaling, is associated with PD-L1 mRNA expression. It was reported that EGFR activation increases PD-L1 expression through p-ERK1/2/p-c-Jun. In addition to promoting PD-L1 transcription, MAPK signaling also stabilizes PD-L1 mRNA by attenuating TPP activity. Since EpCAM is an activator of EGFR signaling, EpCAM might promote escape from immune surveillance. However, EpCAM-mediated immunosuppression and its mechanisms are completely undescribed and remain unclear.

Among all antibody-based immune therapies, anti-PD-1/PD-L1 therapies have the most beneficial outcomes in the treatment of various malignancies. This fact underscores the importance of deeply understanding the processes that control PD-L1 expression. The present disclosure also surprisingly found that EpCAM-mediated PD-L1 stabilization gives rise to escape from immune surveillance through the EpEX-EGFR-ERK signaling axis; EpCAM inhibits antitumor immunity by activating the PD-1/PD-L1 pathway to suppress T-cell function. EpEX increases PD-L1 protein stability rather than mRNA level, while blockade of EGFR or MAPK signaling pathways attenuates EpEX-mediated stabilization of PD-L1 protein. Furthermore, a combination of an anti-PD-L1 antibody and a EpCAM-neutralizing antibody shows enhanced therapeutic efficacy and high levels of tumor-localized CD8+ T cells.

Accordingly, the present disclosure provides a method for treating, inhibiting or eliminating a cancer in a subject, comprising administering to a subject in need thereof, an effective amount of an inhibitor or an antagonist targeting to EGF-like domain I within the EpEX, and an inhibitor or an antagonist targeting to PD-L1 in an amount effective to inhibit expression or activation of PD-L1.

In one embodiment, the inhibitor or an antagonist targeting to PD-L1 is a PD-L1 checkpoint inhibitor.

In some embodiments, PD-L1 checkpoint inhibitors include but are not limited to molecules that decrease, block, inhibit, abrogate or interfere with signal transduction resulting from the interaction of PD-L1 with either one or more of its binding partners, such as PD-1 and B7-1. In some embodiments, a PD-L1 checkpoint inhibitor is a molecule that inhibits the binding of PD-L1 to its binding partners. In some embodiments, the PD-L1 checkpoint inhibitor inhibits binding of PD-L1 to PD-1 and/or B7-1.

Non-limiting examples of the anti-PD-L1 antibody include MEDI4736, atezolizumab, avelumab or durvalumab.

The inhibitor or the antagonist targeting to EGF-like domain I within the EpEX may be present in a combination and/or administered to a patient in a therapeutically effective amount and, in some embodiments, in a therapeutically effective amount that produces a synergy when the inhibitor or an antagonist is administered together with the inhibitor or an antagonist targeting to PD-L1. Such an effective amount may vary according to patient characteristics, including gender, size, age, cancer type, cancer stage, route of administration, patient tolerance, toxicity or side effects, and other factors that a skilled medical practitioner would take into account when establishing appropriate patient dosing.

A synergistic effect of a combination of therapies permits the use of lower dosages of one or more of the therapeutic agents and/or less frequent administration of said therapeutic agents to a patient with a cancer tumor. The ability to utilize lower dosages of therapeutic agents and/or to administer said therapies less frequently reduces the toxicity associated with the administration of said therapies to a subject without reducing the efficacy of said therapies in the treatment of a solid lung cancer tumor. In addition, a synergistic effect can result in improved efficacy of therapeutic agents in the management, treatment, or amelioration of a cancer tumor. The synergistic effect of a combination of therapeutic agents can avoid or reduce adverse or unwanted side effects associated with the use of either single therapy.

Combinations

Also provided is a combination for cancer immunotherapy, comprising an inhibitor or an antagonist targeting to EGF-like domain I within the EpEX, and an inhibitor or an antagonist targeting to PD-L1. In some embodiments, the inhibitor or an antagonist targeting to EGF-like domain I within the EpEX can be formulated together with the inhibitor or an antagonist targeting to PD-L1 or the inhibitor or an antagonist targeting to EGF-like domain I within the EpEX and the inhibitor or an antagonist targeting to PD-L1 can be separately formulated. Each component of a combination can be supplied in a separate, individual container.

In certain non-limiting embodiments, the pharmaceutical combination can be formulated using pharmaceutically acceptable carriers well known in the art that are suitable for oral administration. Such carriers enable the pharmaceutical compositions to be formulated as tablets, pills, capsules, liquids, gels, syrups, slurries, suspensions and the like, for oral or nasal ingestion by a patient to be treated. In certain embodiments, the pharmaceutical combination can be a solid dosage form. In certain embodiments, the tablet can be an immediate release tablet. Alternatively or additionally, the tablet can be an extended or controlled release tablet. In certain embodiments, the solid dosage can include both an immediate release portion and an extended or controlled release portion.

In certain embodiments, the pharmaceutical combinations can be formulated using pharmaceutically acceptable carriers well known in the art that are suitable for parenteral administration. The terms “parenteral administration” and “administered parenterally,” as used herein, refers to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. For example, and not by way of limitation, combinations of the present disclosure can be administered to the patient intravenously in a pharmaceutically acceptable carrier such as physiological saline.

Treatment of Cancers

The methods and combination of the present disclosure can be used to treat a cancer in a subject.

Non-limiting examples of preferred cancers for treatment include melanoma (e.g., metastatic malignant melanoma), renal cancer, prostate cancer, breast cancer, colorectal cancer and lung cancer. Examples of other cancers that may be treated using the methods of the invention include bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, and combinations of said cancers.

In some embodiments, the cancer is an EpCAM-overexpressing cancer, EGFR-overexpressing or activating cancer, AKT-overexpressing or overactivating cancer, MAPK-overexpressing or activating cancer, FOXO3a-inactivating cancer, HtrA2-inactivating cancer or PD-L1 expressing cancer. In a further embodiment, the cancer is a metastatic cancer or an advanced cancer. In a further embodiment, the cancer is a metastatic colorectal cancer or small cell lung cancer or an advanced colorectal cancer or small cell lung cancer.

The combinations described herein can, therefore, be administered to subjects in need thereof as a second, third, fourth, fifth, sixth, or more line of treatment. In one embodiment, the subject is EpCAM-overexpressing. The combinations described herein can be administered to a subject who has been treated with at least one anti-cancer therapy or anti-cancer agent. In certain instances, the subject has received at least one anti-cancer therapy including, for example, chemotherapy, radiotherapy, surgery, targeted therapy, immunotherapy, or a combination thereof. The subject can have a cancer that is resistant/refractory to treatment with at least one anti-cancer agent.

The practice of the present invention employs, unless otherwise indicated, conventional techniques of molecular biology (including recombinant techniques), microbiology, cell biology, biochemistry immunohistochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The present disclosure is further illustrated by the following examples which should not be construed as further limiting. The contents of all figures and all references, patents and published patent applications cited throughout this application are expressly incorporated herein by reference.

### EXAMPLE

Materials and Methods

Chemicals and Antibodies

Antibodies against human EpCAM and p84 were purchased from Abcam, and antibody against β-catenin was from Santa Cruz. Anti-α-tubulin antibody was from Sigma-Aldrich. Polyclonal antibodies detecting total ERK and Thr202/Tyr204-phosphorylated ERK, total AKT and Ser473-phosphorylated AKT, total FOXO3a, Ser253-phosphorylated FOXO3a, Ser318/321-phosphorylated FOXO3a, and The32-phosphorylated FOXO3a, active (non-phospho Ser33/Ser37/Thr41) β-catenin, HtrA2, cytochrome c, COX IV, XIAP, and PD-L1 were from Cell Signaling Technology. U0126 (MEK inhibitor), wortmannin (PI3Kinhibitor) and MG132 (proteasome inhibitor) were obtained from Selleck Chemicals.

Cell Culture

Human lung adenocarcinoma cells (H441), lung carcinoma cells (H460), embryonic kidney cells (HEK293T), colorectal carcinoma cells (HCT116), colorectal adenocarcinoma cells (SW620) were obtained from American Type Culture Collection (ATCC). H441 and H460 were cultured in RPMI 1640 medium (Gibico), and HEK293T, HCT116 and SW620 cells were cultured in DMEM (Gibico). All cells were maintained in conditioned medium supplemented with 10% fetal bovine serum (FBS; Gibco) and 100 μg/ml Penicillin/Streptomycin (P/S; Gibco) at 37° C. in a humidified incubator with 5% CO₂.

Western Blotting

Cells were lysed with RIPA buffer (20 mM Tris-HCl, pH7.4, 150 mM NaCl, 1 mM EDTA, 1 mM EGTA, 1% Nonidet P-40, 1% sodium deoxycholate, 2.5 mM sodium pyrophosphate) containing protease inhibitor (Roche) and phosphatase inhibitor (Roche) to extract whole cell lysates. The lysates were then quantified using the Pierce™ BCA Protein Assay Kit (Thermo). The lysates were mixed with 5× sample buffer (50 mM Tris-HCl, pH 6.8, 2% SDS, β-mercaptoethanol, 0.1% bromophenol blue, 10% glycerol), separated by SDS-polyacrylamide gel electrophoresis (SDS-PAGE), and transferred to PVDF membrane (Millipore). Nonspecific antibody-binding sites on the PVDF membrane were blocked with 3% Bovine serum albumin (BSA) in TBS-T (TBS buffer containing 0.1% Tween 20) and membranes were incubated with indicated antibody overnight at 4° C., followed by incubation with HRP-conjugated secondary antibodies (Jackson ImmunoResearch Laboratories) at room temperature (RT) for 1h. The protein bands were subsequently visualized with chemiluminescence reagents (Millipore) and detected by UVP BioSpectrum 600 Imagine (UVP). The protein expression was quantified by Gel-Proanalyzer 3.1 (Media Cybernetics).

Production and Purification of EpEX-6×his Recombinant Protein

The DNA fragment encoding EpEX (amino acids 24-262 of EpCAM) was amplified by PCR using PfuTurbo DNA polymerase and cloned into pSecTag2 vector with C-terminal 6×His tag to generate pSecTag2-EpEX-6×His. The EpEX-6×His fusion protein was produced with Expi293F™ Expression System (Thermo) and purified by Ni-affinity column (GE healthcare).

Extracellular Interactions Between EpEX-Fc and EGFR

HCT116 cells were harvested with 10 mM EDTA in PBS, and incubated with EpEX-Fc for 1 h at 4° C. After incubation, 2 mM DTSSP (Thermo) was used as a cross-linker to stabilize the interaction between EpEX-Fc and EGFR. To stop the cross-linking reaction, Tris, pH 7.5 was added to a final concentration of 20 mM. Membrane proteins were extracted using Mem-PER Eukaryotic Membrane Protein Extraction Reagent Kit (Thermo). Finally, the EpEX-Fc-EGFR complex was pulled-down with Dynabeads® Protein G (Invitrogen), and probed by Western blotting.

Construction of the EpCAM EGF-Like Domain Deletion

In its extracellular domain, EpCAM contains two EGF-like domains at amino acids 27-59 (1^{st} EGF-like domain) and 66-135 (2^{nd} EGF-like domain), and a cysteine-free motif (Schnell U, Kuipers J, Giepmans B N. EpCAM proteolysis: new fragments with distinct functions? Bioscience reports 2013; 33:e00030). The EpCAM EGF-like domain deletion was generated using the standard QuikChange™ deletion mutation system with 1^{st} forward mutagenic deletion primer (5′-GCAGCTCAGGAAGAATCAAAGCTGGCTGCC-3′) (SEQ ID NO: 2), 1^{st} reverse mutagenic deletion primer (5′-GGCAGCCAGCTTTGATTCTTCCTGAGCTGC-3′) (SEQ ID NO: 3), 2^{nd} forward primer (5′-AAGCTGGCTGCCAAATCTGAGCGAGTGAGA-3′) (SEQ ID NO: 4) and 2^{nd} reverse primer (5′-TCTCACTCGCTCAGATTTGGCAGCCAGCTT-3′) (SEQ ID NO: 5). The PCR amplifications were performed using KAPA HiFi Hot Start DNA polymerase (Kapa Biosystems), and products were treated with restriction enzyme, DpnI (Thermo Scientific), to digest methylated parental DNAs.

Cycloheximide Chase Assay

Cycloheximide, a protein synthesis inhibitor, was used to evaluate the stability of PD-L1. Cells were treated with cycloheximide for 0, 2, 4 or 6 h. Proteins were extracted and Western blot was performed to detect PD-L1 protein level.

Immunoprecipitation Assay

Cells were lysed in lysis buffer (50 mM Tris-HCl, pH 7.4, 150 mM NaCl, 1% NP-40) with protease inhibitors (Roche). For immunoprecipitation (IP), cell lysates were incubated with antibodies for 6 h at 4° C. Then, 20 μl Dynabeads® Protein G was added, and the mixture was incubated for 2 h at 4° C. to pull-down the antibody-bound protein. The IP samples were washed with PBS three times, denatured in sample buffer, and analyzed by Western blotting.

Generation of Monoclonal Antibodies and Purification of IgG

Generation of EpAb2-6 and control IgG were performed as described previously (Liao M Y, Lai J K, Kuo M Y, Lu R M, Lin C W, Cheng P C, et al. An anti-EpCAM antibody EpAb2-6 for the treatment of colon cancer. Oncotarget 2015; 6:24947-68). The protocol was approved by the Committee on the Ethics of Animal Experiments of Academia Sinica (AS IACUC: 11-04-166).

Lentivirus-Mediated Short Hairpin RNA (shRNA) Knockdown

All lentiviral shRNA constructs were purchased from the RNAi Core Facility at Academia Sinica (Taipei, Taiwan). Lentiviral production, infection, and selection were performed according to protocols from the RNAi Consortium (Academia Sinica, Taiwan). To produce the lentivirus, HEK293T cells were transiently co-transfected with shRNA plasmid, packaging (pCMV-AR8.91) and envelope (pMD.G) expression plasmids using PolyJet DNA Transfection Reagent (SignaGen Laboratories). The next day, the transfection solution was replaced with 1% BSA-containing medium to improve virus yield. Two days after transfection, medium containing lentivirus was collected. Cells were cultured with lentivirus-containing medium, supplemented with 8 μg/ml polybrene for another 48 h. The transduced cells were selected with 2 μg/ml puromycin for 4 days and the knockdown efficiency was measured by Western blotting. The target sequence for human EpCAM-specific shRNA was shRNA, 5′-GCAAATGGACACAAATTACAA-3′ (SEQ ID NO: 1). Luciferase shRNA (shLuc) was used as a negative control.

RNA Extraction, cDNA Synthesis, Quantitative Reverse Transcription Polymerase Chain Reaction (qRT-PCR)

Total RNA extraction, first strand cDNA synthesis, and SYBR-green based real-time PCR were performed as described in the manufacturer's instructions. To extract total RNA, cells were lysed using TRIzol reagent (Invitrogen), and proteins and phenol were removed from TRIzol using chloroform. After centrifugation, the top colorless layer was collected and mixed with isopropanol to precipitate RNA pellet. The RNA pellet then was washed with 70% ethanol, air-dried at room temperature, and dissolved in RNase free water. For first strand cDNA synthesis, 5 μg of total RNA was used for reverse transcription with oligo (dT) primer and SuperScriptIII reverse transcriptase (Invitrogen) at 50° C. for 60 min. Target gene levels were evaluated by quantitative PCR (qPCR), using LightCycler 480 SYBR Green I Master Mix (Roche) and a LightCycler480 System (Roche). GAPDH mRNA expression was measured as endogenous housekeeping control to normalize all qPCR reactions. The qPCR reaction was 95° C. for 5 min, followed by 40 cycles of denaturation at 95° C. for 10 s, annealing at 60° C. for 10 s and extension at 72° C. for 30 s. Final results were calculated from three independent experiments.

Immunofluorescence Assay

For immunofluorescence, 2×10⁴ cells were seeded on 12-mm cover slides in 24-well culture plate, fixed with 2% paraformaldehyde and incubated with 0.1% Triton X-100 to increase the permeability of cell membrane. Then, samples were blocked with 1% BSA for 1 h at room temperature and stained with primary antibodies at 4° C. overnight. Slides were washed and stained with FITC or Alexa568-conjugated secondary antibodies and DAPI for 40 min. Cover slides were then wash with PBS and mounted in mounting solution (Vector Laboratories). The slides were examined with a confocal microscope (Leica TCS-4 SP5).

Apoptosis Protein Array

HCT116 cells were stimulated with 20 μg/ml EpAb2-6 for 6 h, and apoptosis arrays were performed according to the manufacturer's instructions (R&D System; ARY009). The membranes (array) were detected by the UVP BioSpectrum 600 Imagine (UVP). The arrays were quantified by a Gel-Pro analyzer 3.1 (Media Cybernetics, Inc.).

Flow Cytometry Analysis

Cells were harvested, washed and suspended in FACS buffer (PBS with 1% FBS), and 10⁵ cells were transferred to 96-well shrill-based plates. Cells were stained with different antibodies for 1 h at 4° C., and subsequently stained with indicated hycoerythrin (PE)-conjugated secondary antibodies for 1 h at 4° C. Then, cells were washed with FACS buffer twice and suspended in 400 μl FACS buffer. Florescence signals were analyzed using flow cytometry (BD FASCSC Anto™II) and measured with FCS Express V3 software. The data were collected from three independent experiments.

Chromatin Immunoprecipitation

In brief, HCT116 cells (1×10⁵) were treated with 20 μg/ml EpAb2-6 for 6 h, followed by crosslinking fixation in 1% formaldehyde. Fixation was quenched by the addition of glycine to a final concentration of 200 mM and fixed chromatin complexes were then sonicated to an average length of 250 base pairs using an MISONIX Sonicator 3000. The sonicated protein-DNA complexes were subjected to immunoprecipitation using 2 μg of antibodies against FOXO3a. The immunoprecipitated DNA was recovered by a PCR purification kit (Qiagen), and the amount of target DNA was detected by PCR.

Dual-Luciferase Reporter Assays

The human HtrA2 proximal promoter fragment covering the region −1628 to +86 and −700 to +86 (with the transcriptional start site denoted as +1) were PCR amplified and then cloned into the firefly luciferase reporter plasmid pGL4.18 vector (Promega). To assess the effect of EpA2-6 on HtrA2 promoter activity, HCT116 and SW620 cells (1×10⁴/well) seeded onto 24-well dishes were transiently transfected with HtrA2 promoter reporter constructs in combination with a plasmid expressing Renilla luciferase using the PolyJet transfection reagent (SignaGen Laboratories, USA) for 24 h, followed by 20 μg/ml EpAb2-6 for 6 h stimulation. Cell lysates were prepared and subjected to the luciferase activity assay using the Dual-Luciferase Reporter assay kit (Promega, USA). Firefly luciferase activity was normalized to Renilla luciferase activity, and final data is presented as the fold induction of luciferase activity compared to EpAb2-6-IgG controls. Data are expressed as means±SD from three independent experiments.

Apoptosis and Mitochondrial Membrane Potential Assay

Cells (2×10⁵) were seeded onto 24-well dishes and treated with 20 μg/ml control IgG or EpAb2-6 for 6 h. The apoptotic cell and mitochondrial membrane potential were detected using FITC Annexin V apoptosis detection kit (BD Biosciences) and MitoStatus Red (BD Biosciences), respectively. The apoptotic cell and mitochondrial membrane potential were analyzed using a flow cytometer (Thermo Fisher Scientific). Each measurement was carried out at least three times to ensure reproducibility. The effect of gene knockdown on EpAb2-6-induced apoptosis was supplementary material Table 1.

TABLE 1
The effect of gene knockdown on EpAb2-6-induced apoptosis

| | A | B | A − B | C (A − B/A) |
|---|---|---|---|---|
| shEGFR | 35.9 | 17.4 | 18.5 | 51.5 |
| shc-Met | 37.1 | 28.2 | 8.9 | 24.0 |
| shAKT | 37.1 | 23.4 | 13.7 | 36.9 |
| shFOXO3a | 37.2 | 25.7 | 11.5 | 30.9 |
| shHtrA2 | 47.7 | 29.3 | 18.4 | 38.6 |

A: EpAb2-6-induced apoptosis in control knockdown cells (%)
B: EpAb2-6-induced apoptosis in indicated knockdown cells (%)
A − B: The decrease of EpAb2-6-induced apoptosis in indicated knockdown cells (%)
C: The decrease of EpAb2-6-induced apoptosis in indicated knockdown cells compared to apoptosis in control knockdown cells (%), (A − B/A)

Immunohistochemistry

Human lung cancer tissue microarray was purchased from Super BioChip. After exhausting the endogenous hydroperoxidases with 3% hydrogen peroxide in methanol for 30 min, the tissue microarray was blocked with 1% BSA for 1 h and exposed to anti-PD-L1 (clone 28-2, abcam) or EpAb3-5 (Liao M Y, Lai J K, Kuo M Y, Lu R M, Lin C W, Cheng P C, et al. An anti-EpCAM antibody EpAb2-6 for the treatment of colon cancer. Oncotarget 2015; 6:24947-68) at 4° C. overnight. After washing with PBST, the tissue microarray was processed using the standard procedure of Super Sentivie™ IHC Detection System (BioGenex) and stained by DAB. Nuclei were stained with Mayer's hematoxylin solution (Wako).

PBMC and T Cell Isolation and Culture

For the experiments using PBMCs, blood samples were drawn from healthy donors and collected into 10 ml Vacutainer® tubes containing the anticoagulant EDTA (BD Bioscience). After centrifugation at 1500 rpm for 10 min, plasma was removed from the sample and mixed with an equal volume of PBS. The mixture was layered on Ficoll-Paque plus (Ficoll:Blood=1:2) and centrifuged at 1500 rpm for 30 min. The PBMC layer (buffy coat) was collected and washed with PBS with 0.5% BSA, 2 mM EDTA twice. To obtain purified CD3⁺ T cells, PBMCs were positively selected by anti-CD3 magnetic beads (MACS), according to the standard protocol. Isolated CD3⁺ T cells (10⁶) were cultured and activated by 25 μl anti-CD3/anti-CD28-coated dynabeads (Invitrogen) in RPMI 1640 supplemented with 10% FBS, 100 μg/ml P/S, 12.5 ng/ml IL-2 (Gibico), and 1 ng/ml IL-15 (MACS) for 48 h. The protocol was approved by the Institutional Review Board of Academia Sinica (AS IRB: AS-IRB01-19049).

Cell Viability Assays In Vivo

NOD/SCID mice were injected intravenously with HCT116-GFP cells; mice bearing circulating HCT116-GFP cells were intravenously treated with EpAb2-6 or an equivalent dosage of control IgG at 1 h after cell injection (antibody was delivered at 20 mg/kg). Then, blood samples were obtained from the facial vein of mice and the fluorescence intensity of whole blood was quantified at the indicated time points. The fluorescence was measured with a microplate reader (Molecular Devices, SpectraMax M5) at an excitation wavelength of 355 nm and emission wavelengths of 440 nm.

Subcutaneous Human Colorectal Cancer Studies

Subcutaneous studies were performed as previously reported (Liao M Y, Lai J K, Kuo M Y, Lu R M, Lin C W, Cheng P C, et al. An anti-EpCAM antibody EpAb2-6 for the treatment of colon cancer. Oncotarget 2015; 6:24947-68). The protocol was approved by the Committee on the Ethics of Animal Experiments of Academia Sinica (AS IACUC: 11-04-166).

Orthotopic Implantation and Therapeutic Studies

Orthotopic studies were performed as previously reported (Wu C H, Kuo Y H, Hong R L, Wu H C. alpha-Enolase-binding peptide enhances drug delivery efficiency and therapeutic efficacy against colorectal cancer. Sci Transl 7 Med 2015; 7:290ra91). Briefly, NSG mice (NOD.Cg-Prkdcscid Il2rgtm1Wj1/SzJ) were used for orthotopic implantation of HCT116 cells, which were previously infected with Lenti-luc virus (lentivirus containing luciferase genes). The mice were anesthetized by i.p. injection of Avertin, 2,2,2-Tribromo-ethanol (Sigma-Aldrich) at a dose of 250 mg/kg. Tumor development was monitored by bioluminescence imaging. For the orthotopic therapeutic study, tumor-bearing mice were treated with control IgG or EpAb2-6 (20 mg/kg). Tumor progression was monitored by quantification of bioluminescence. Mouse body weight and survival rate were measured. Animal care was carried out in accordance with the guidelines of Academia Sinica, Taiwan. The protocol was approved by the Committee on the Ethics of Animal Experiments of Academia Sinica (AS IACUC: 11-04-166).

Gene Set Enrichment Analysis, GSEA

The gene expression matrix was obtained from LUAD projects in TCGA. The 25% of samples with highest EpCAM expression were defined as the ‘EpCAM High’ group, and the 25% with least EpCAM expression were defined as the ‘EpCAM Low’ group. T cells activation and T cells proliferation gene sets provided by GSEA website were then used to analyze the data.

Statistical Analyses

All data are represented as means±standard deviation (SD) from at least three independent experiments. Significant differences from the respective control for each experimental condition were calculated using Student's t-test unless otherwise specified. P-values <0.05 (*), <0.01 (**) or <0.001 (***) are indicated as significant. Survival analysis was performed using a log-rank test. The correlation coefficient was calculated using Spearman's-analysis.

### Example 1 EpEX Binds to EGFR Through its EGF-Like Domain I

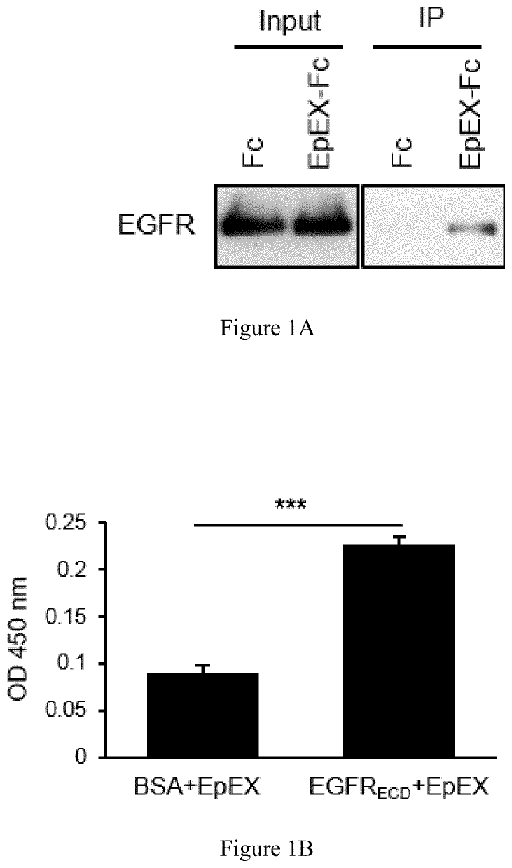

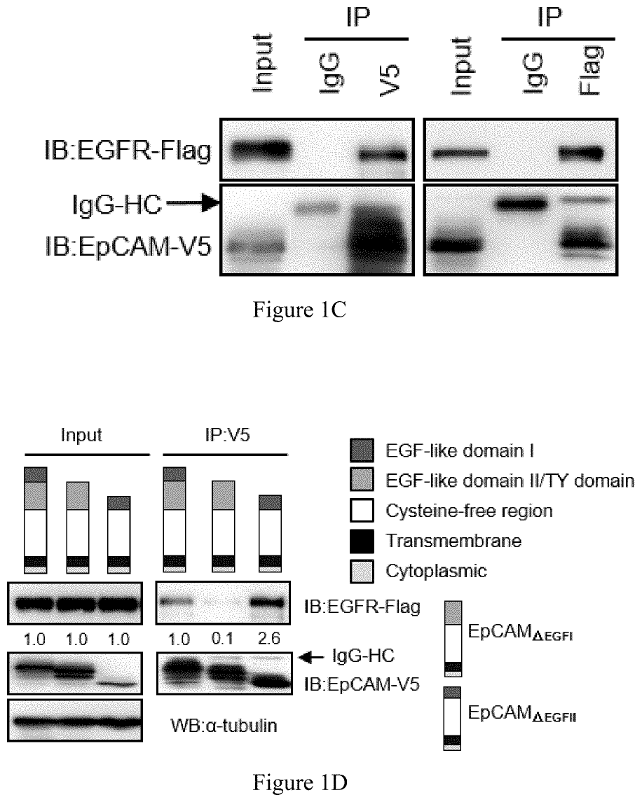

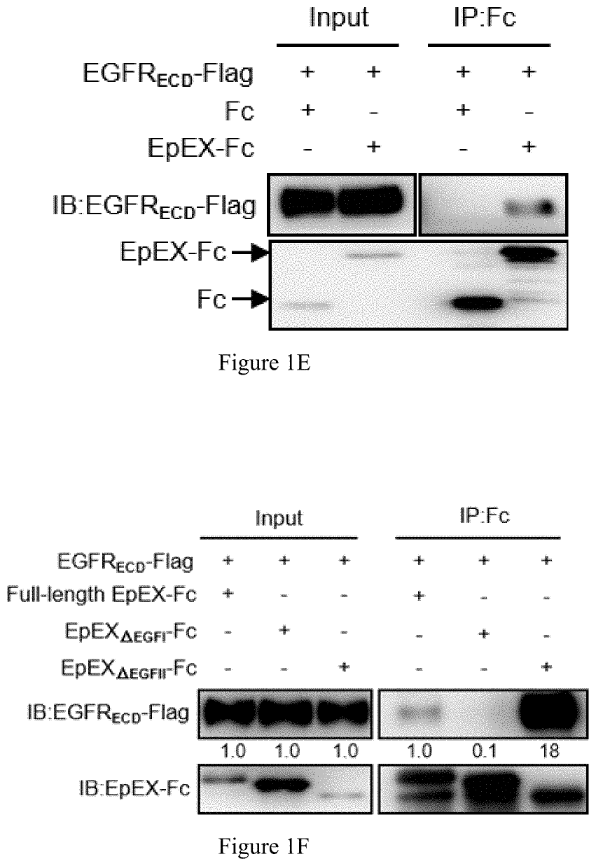

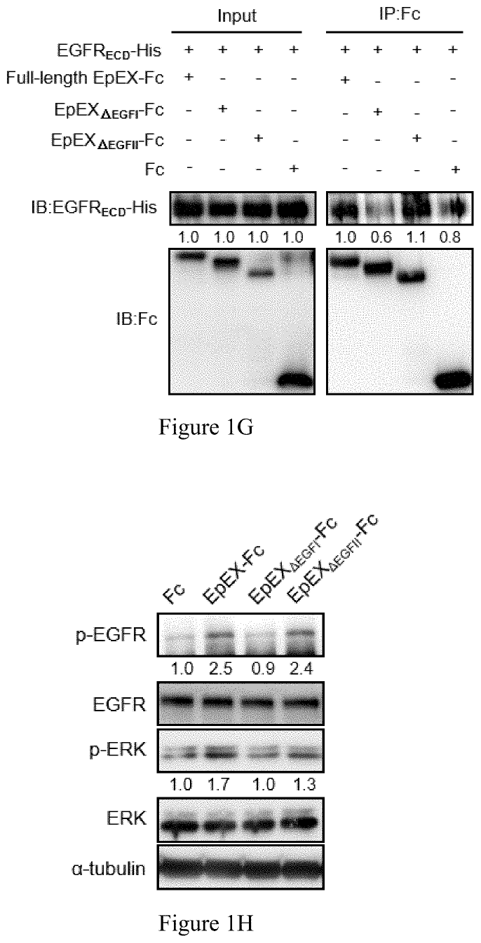

To verify that EpEX interacts with EGFR, we used the cross-linker, DTSSP, to stabilize the EpEX-EGFR complex. The binding of EpEX to EGFR was confirmed by IP and Western blotting (FIG. 1A). To evaluate the direct binding of recombinant EpEX to the extracellular domain of EGFR (EGFR_{ECD}), we performed ELISA to probe the direct interaction between purified EpEX and EGFR_{ECD} protein (FIG. 1B).

To test whether membrane-bound EpCAM could bind to EGFR, we performed immunoprecipitation experiments using HEK293T cells with EpCAM-V5 and EGFR-Flag overexpression. The interaction between exogenous EpCAM and EGFR was detected by co-IP (FIG. 1C). In EpCAM extracellular domain, it contains two EGF-like domains at amino acids 27-59 (1^{st} EGF-like domain) and 66-135 (2^{nd} EGF-like domain), and a cysteine-free motif. To identify the specific region of EpEX that binds to EGFR, we constructed different EGF-like domain-deleted EpCAM mutants. Surprisingly, the EGF-like domain I-deleted EpCAM mutant (EpCAM_{ΔEGFI}) showed decreased binding to EGFR, while the EGF-like domain II-deleted EpCAM mutant (EpCAM_{ΔEGFI}) exhibited an enhanced interaction with EGFR (FIG. 1D). To further evaluate the binding of soluble EpEX to EGFR_{ECD}, HEK293T cells were co-transfected with different vectors expressing soluble EGFR_{ECD}-Flag or EpEX-Fc, and the protein complex was examined in culture media (FIG. 1E). Soluble EGF-like domain II-deleted EpEX-Fc (EpEX_{ΔEGFII}-Fc) showed significantly increased affinity, while EGF-like domain I-deleted EpEX-Fc (EpEX_{ΔEGFI}-Fc) exhibited decreased affinity for EGFR_{ECD}-Flag compared to EpEX-Fc (FIG. 1F). Overall, we found that the membrane-bound EpCAM and secreted EpEX both bind EGFR and deletion of the EGF domain I diminished this binding (FIGS. 1C-1F).

Similar results were observed using purified wild-type or mutant EpEX and EGFR_{ECD} recombinant proteins. The recombinant EpEX_{ΔEGFII} protein had a stronger binding affinity for EGFR_{ECD} than wild-type controls, and the EpEX_{ΔEGFI} protein lost its ability to bind EGFR_{ECD} (FIG. 1G). Moreover, The EpEX_{ΔEGFII} protein induced EGFR signaling like the wild-type control, but EpEX_{ΔEGFI} protein did not (FIG. 1H). These results suggest that the EGF-like domain I in EpEX is the major domain responsible for the EpEX-EGFR interaction and activation of EGFR signaling.

### Example 2 Inhibiting EpEX Increases FOXO3a Nuclear Accumulation and Induces Apoptosis

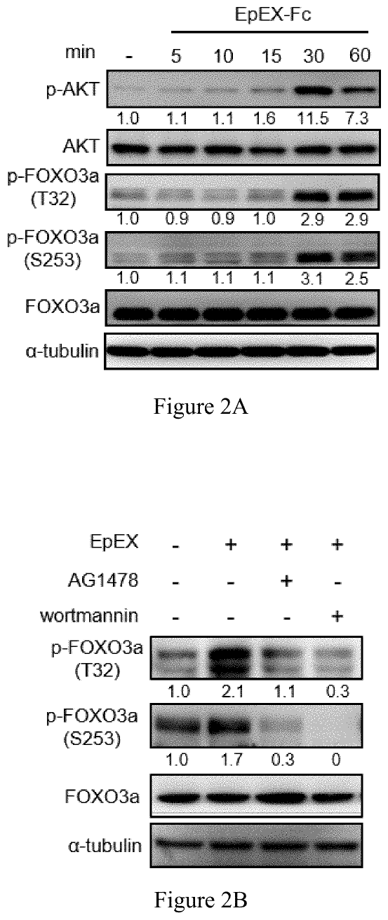

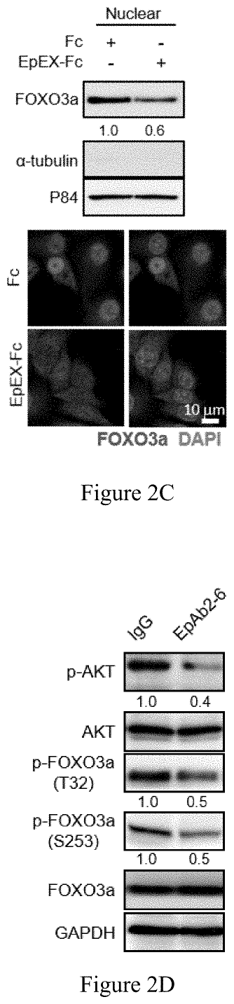

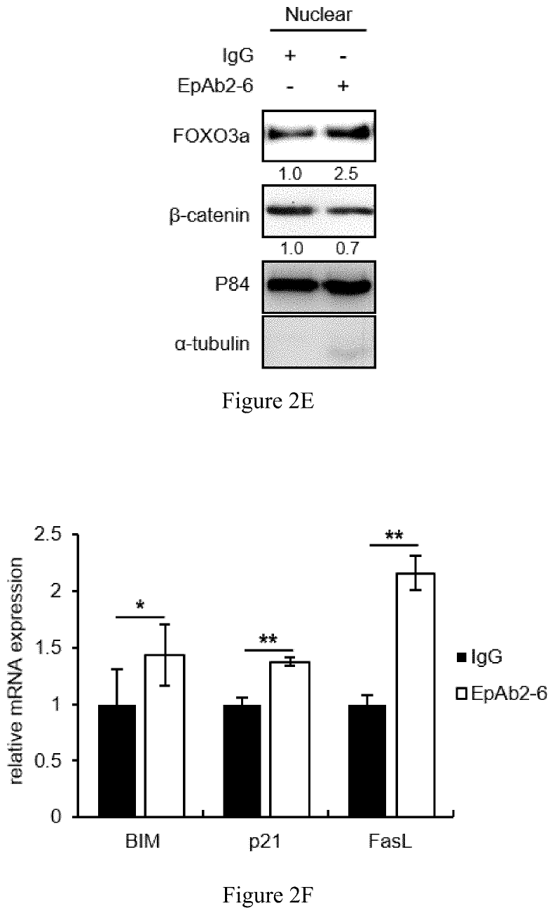

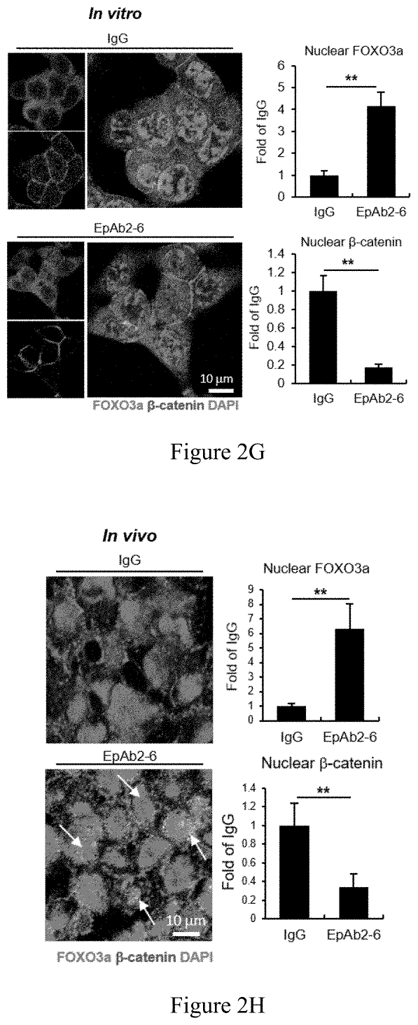

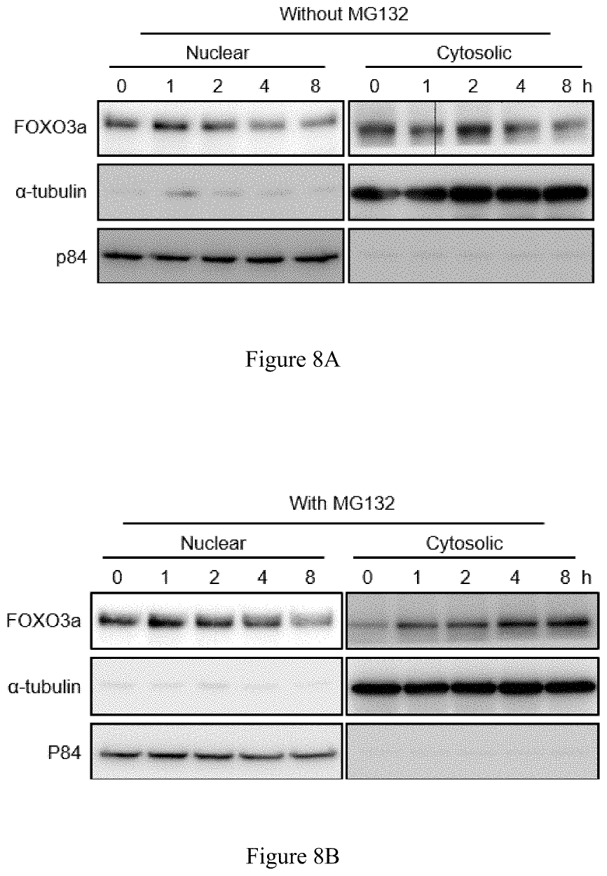

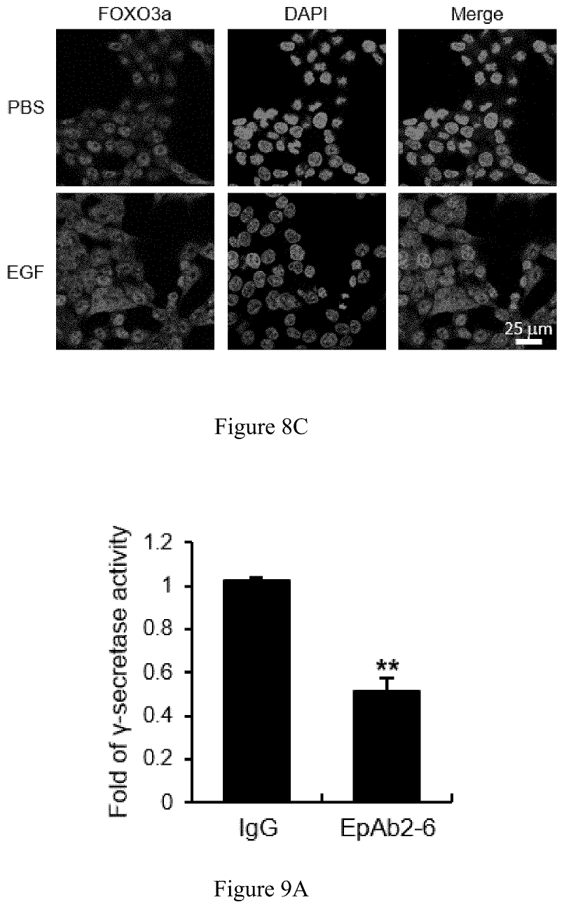

Since we found that EpEX activates EGFR through EpEX domain I, we further investigated whether EpEX-mediated EGFR activation is important for cancer progression. AKT is a major downstream effector of EGFR, which promotes cell survival partially by inactivating FOXO3a. Inhibition of AKT/FOXO3a signaling has been suggested an essential step in apoptosis and could inhibit tumorsphere formation of the SKOV3 ovarian cancer cell line. To evaluate whether EpEX affects FOXO3a function, we examined FOXO3a phosphorylation and intracellular location. Immunoblotting analysis demonstrated that EpEX induced the phosphorylation of AKT and FOXO3a in a time-dependent manner (FIG. 2A), and EpEX-induced phosphorylation of FOXO3a was inhibited by AG1478 (EGFR inhibitor) or wortmannin (PI3K inhibitor) (FIG. 2B). Moreover, after EpEX treatment, FOXO3a translocated from the nucleus to the cytoplasm (FIG. 2C). Because of the EpEX acted through EGFR, we used EGF, a well-known ligand for EGFR, as an inducer. Similar to EpEX treatment, EGF induced nuclear exclusion of FOXO3a (FIGS. 8A-8C). Therefore, we conclude that EpEX inhibits the FOXO3a activity through the EGFR-AKT pathway.

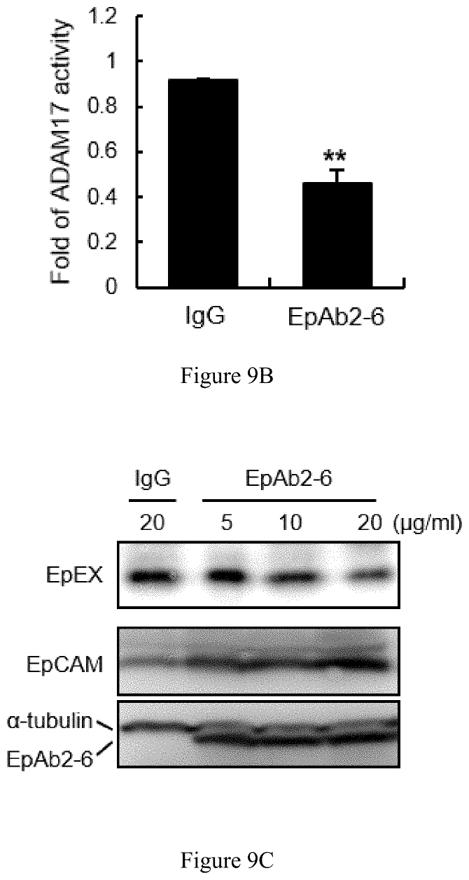

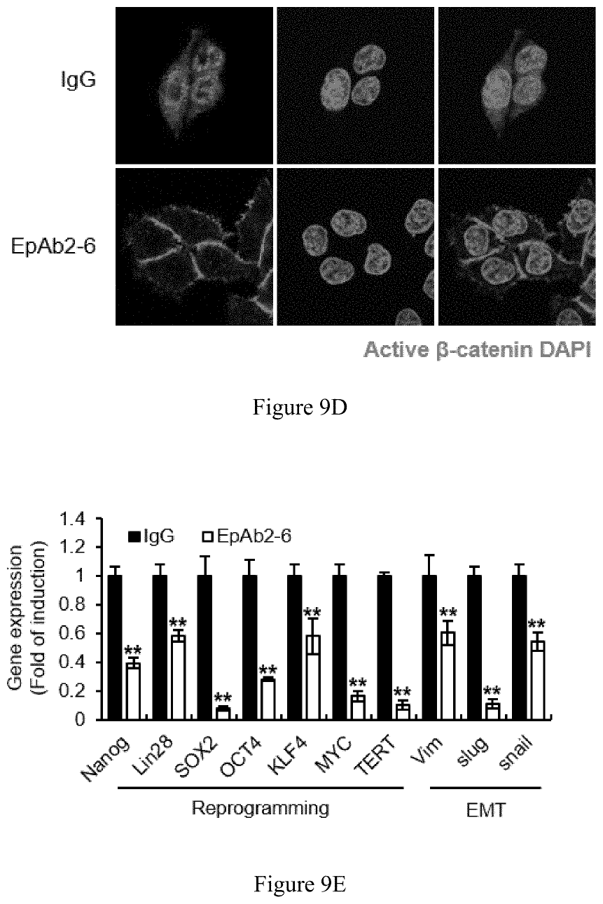

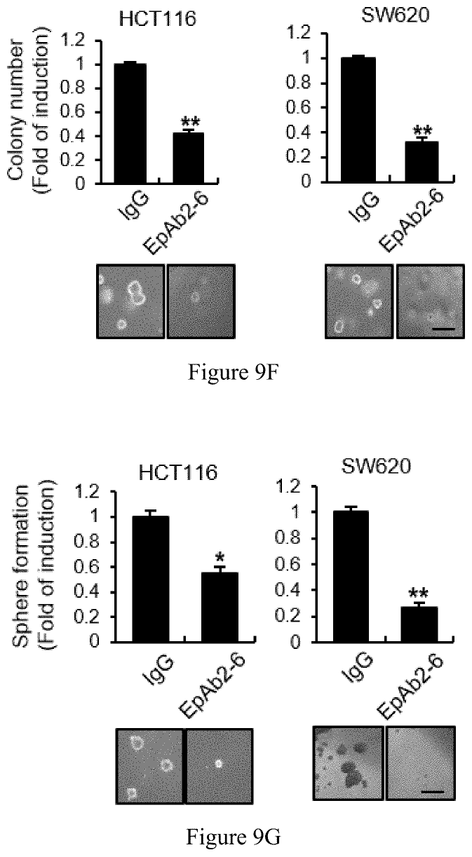

Previously, we had developed a neutralizing antibody, EpAb2-6, which targets EpEX and induces apoptosis (Liang K H, Tso H C, Hung S H, Kuan, II, Lai J K, Ke F Y, et al. Extracellular domain of EpCAM enhances tumor progression through EGFR signaling in colon cancer 16 cells. Cancer Lett 2018; 433:165-755; Liao M Y, Lai J K, Kuo M Y, Lu RAI, Lin C W, Cheng P C, et al. An anti-EpCAM antibody EpAb2-6 for the treatment of colon cancer. Oncotarget 2015; 6:24947-68). EpAb2-6 was employed to block the EpEX. By blocking the function of EpEX, EpAb2-6 treatment is known to interrupt the EpEX/EGFR/ADAM17 axis, which represents a positive feedback loop promoting EpCAM cleavage and subsequently increases EpEX and EpICD production (Liang K H, Tso H C, Hung S H, Kuan, I I, Lai J K, Ke F Y, et al. Extracellular domain of EpCAM enhances tumor progression through EGFR signaling in colon cancer 16 cells. Cancer Lett 2018; 433:165-75). Indeed, decreases in ADAM17 and γ-secretase activity and soluble EpEX release were observed after EpAb2-6 treatment (FIGS. 9A-9C). Moreover, nuclear activated β-catenin and downstream genes, including reprogramming genes and EMT-related genes, were also decreased in EpAb2-6-treated cells (FIGS. 9D and 9E). Next, we examined whether EpCAM contributes to anchorage-independent growth. For this purpose, a soft agar colony formation assay was performed with colon cancer cell lines. EpAb2-6-treated cells formed significantly smaller and fewer colonies compared to the control cells (FIG. 9F). We further cultivated these attached colon cancer cells into tumorspheres and found that EpAb2-6 treatment inhibited tumorsphere formation (FIG. 9G). These results suggest that targeting EpCAM with a specific antibody, EpAb2-6, is a potentially viable strategy to suppress colon cancer malignancy.

Nuclear accumulation of FOXO3a promotes cancer cell apoptosis after treatment with various chemotherapy drugs or EGFR inhibitors. Thus, we wanted to clarify whether inhibiting the function of EpEX facilitates nuclear FOXO3a accumulation. Blocking the function of EpEX with EpAb2-6 treatment also decreased the phosphorylation of AKT and FOXO3a (FIG. 2D) and subsequently increased nuclear accumulation of FOXO3a, while diminishing the nuclear translocation of β-catenin (FIG. 2E). The downstream genes of FOXO3a (BIM, p21, and FASL) were also upregulated in HCT116 cells with EpAb2-6 treatment compared to IgG control (FIG. 2F). Using immunofluorescence staining, FOXO3a and β-catenin were detected in both the cytoplasm and nucleus of colon cancer cells. However, after EpAb2-6 treatment, FOXO3a was increased in the nucleus, but nuclear 3-catenin was decreased both in vitro (FIG. 2G) and in vivo (FIG. 2H). Thus, inhibiting EpEX enhances the nuclear translocation of FOXO3a and consequently upregulates downstream genes, which are involved in promoting apoptosis.

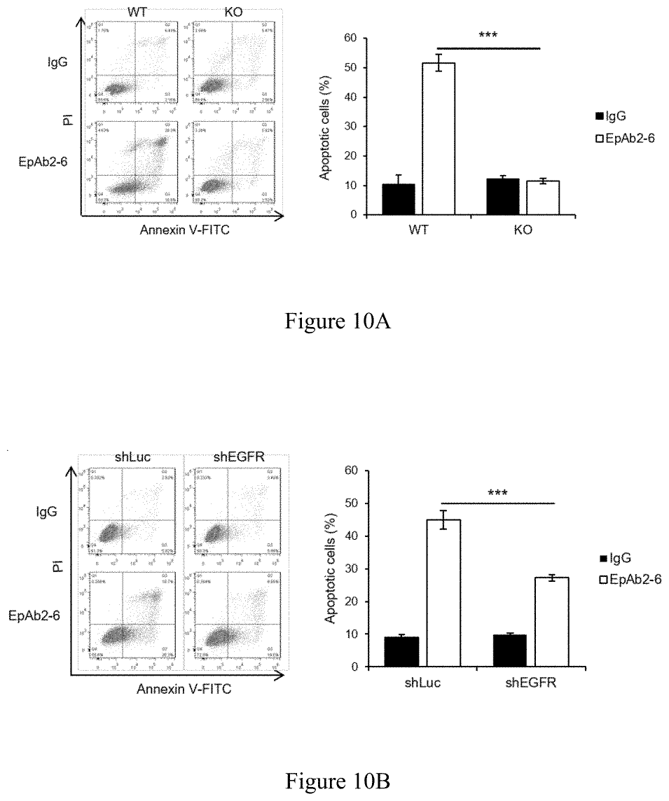

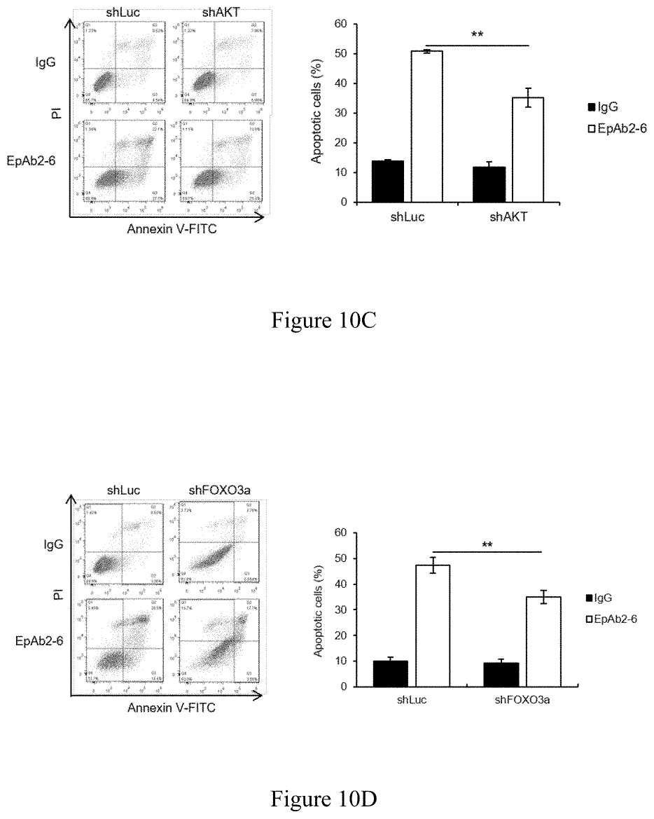

To evaluate whether EpAb2-6 induces apoptosis via EGFR/AKT/FOXO3a, we used shRNA to knockdown EGFR, AKT and FOXO3a expression in colon cancer cells and examined the effect of EpAb2-6 on cancer cell apoptosis. EGFR-, AKT- and FOXO3a-knockdown cells were all less sensitive to EpAb2-6-induced apoptosis than control cells, and EpAb2-6-induced apoptosis was completely abrogated in EpCAM-knockout (KO) cells (FIGS. 10A-10D). Together, our data showed that EpEX induces phosphorylation of FOXO3a and inactivates its biological function; meanwhile, blocking the function of EpEX inhibits the phosphorylation of FOXO3a to promote nuclear accumulation of FOXO3a and activate expression of its downstream pro-apoptotic genes.

### Example 3 HtrA2 Acts Downstream of FOXO3a and Contributes to EpAb2-6-Induced Apoptosis

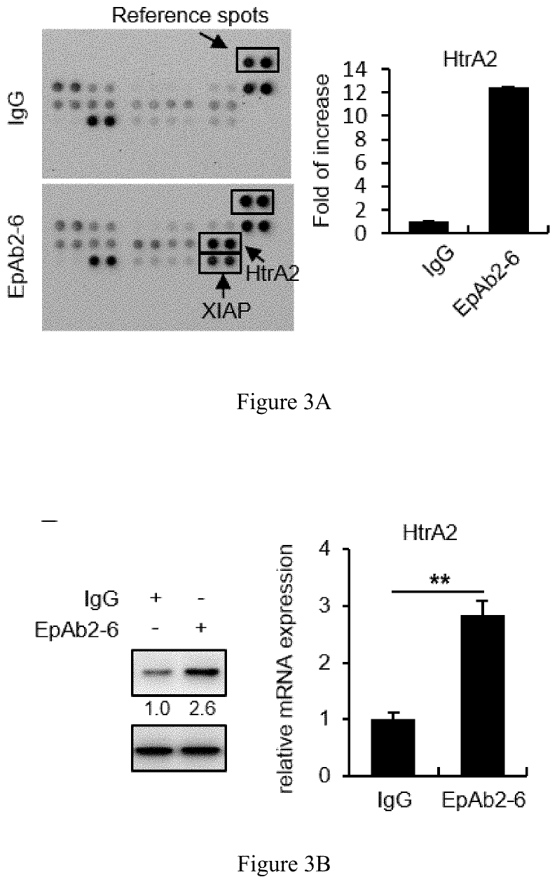

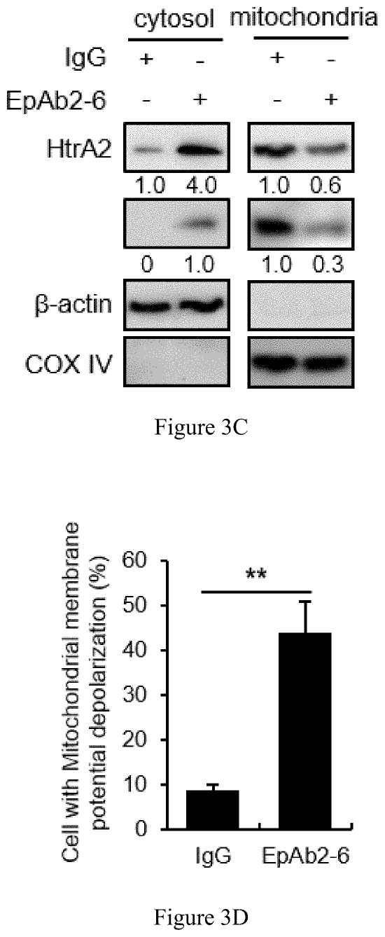

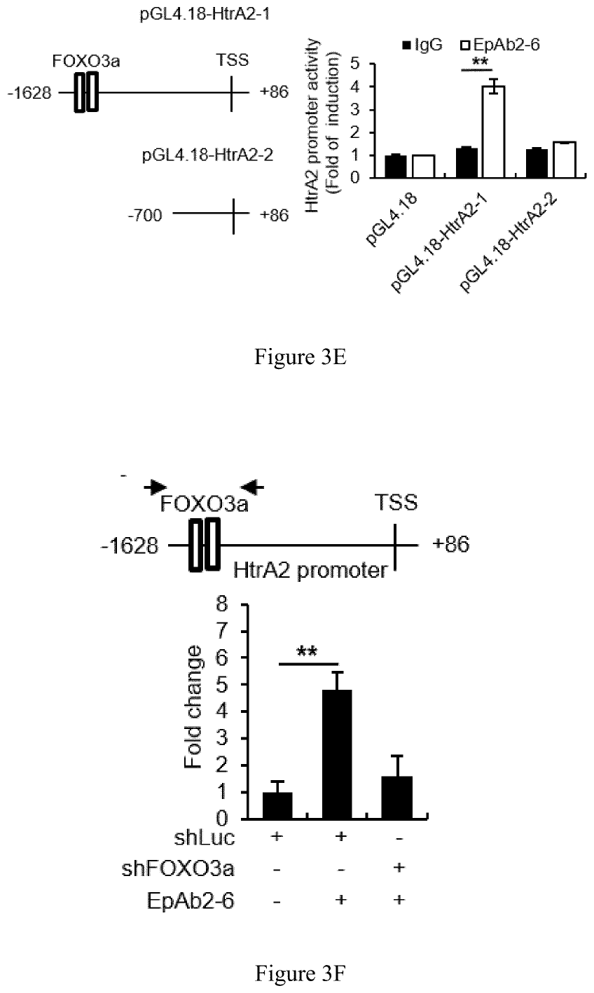

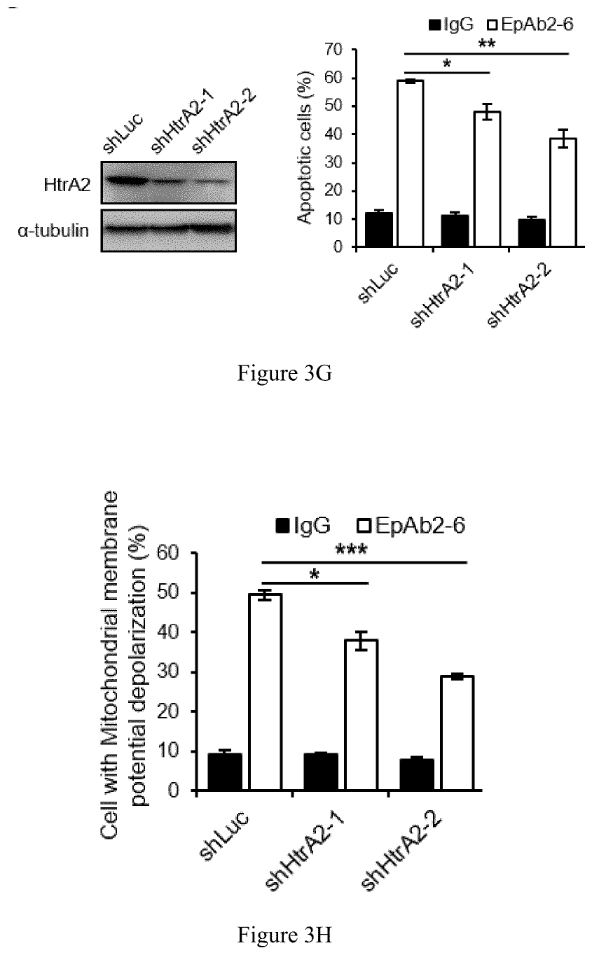

To gain further insight into the mechanisms of EpAb2-6-induced apoptosis, we compared the expression of 35 apoptosis-related signaling proteins in control IgG and EpAb2-6-treated colon cancer cells. We found that among the 35 apoptosis-related proteins, two showed substantial increases in expression after EpAb2-6 treatment compared to the IgG control. These proteins were High temperature requirement A2 (HtrA2) and X-linked inhibitor of apoptosis protein (XIAP) (FIG. 3A).

Immunoblotting and qPCR experiments showed that EpAb2-6 treatment enhanced the protein and mRNA expression of HtrA2 in HCT116 cells (FIG. 3B). According to previous studies, HtrA2 can be released into the cytosol, where it contributes to apoptosis (Bhuiyan M S, Fukunaga K. Mitochondrial serine protease HtrA2/Omi as a potential therapeutic target. Curr Drug Targets 2009; 10:372-83). As expected, we found that EpAb2-6 induced the release of cytochrome c and HtrA2 from mitochondria into the cytosol (FIG. 3C). Furthermore, EpAb2-6 treatment induced the dissipation of the mitochondrial membrane potential (FIG. 3D).

HtrA2 mRNA levels were increased after EpAb2-6 treatment, implying transcriptional regulation of HtrA2 gene was induced by EpAb2-6. To examine this hypothesis, HCT116 cells were transiently transfected with a reporter for the HtrA2 promoter; a region encompassing 1704 bases upstream of the HtrA2 translational start site was used to drive the expression of the firefly luciferase gene (pGL4.18-HtrA2-1) (FIG. 3E). EpAb2-6 treatment led to an approximate 4-fold induction in HtrA2 promoter activity, compared to the IgG control, indicating that the HtrA2 gene is indeed a transcriptional target of EpAb2-6-induced signaling (FIG. 3E). To further elucidate the molecular mechanism controlling HtrA2 gene transcription, the HtrA2 promoter sequence was analyzed using the PROMO virtual laboratory website. This analysis uncovered putative cis-acting response elements (−1283 to −1299) for FOXO transcription factors. Interestingly, to our knowledge, there is no previously published evidence showing that FOXO3a controls HtrA2 promoter activity.

Thus, we examined whether HtrA2 promoter activity was regulated by FOXO3a by generating a reporter construct without the FOXO3a response element (pGL4.18-HtrA2-2), as depicted in FIG. 3E. Indeed, the EpAb2-6-induced increase in luciferase activity was abolished in the absence of a FOXO3a response element (FIG. 3E).

To further elucidate whether FOXO3a directly regulates the HtrA2 promoter, chromatin immunoprecipitation (ChIP) analysis was performed using primers flanking the putative FOXO3a response elements (−1283 to −1299). FOXO3a occupied the HtrA2 promoter after EpAb2-6 treatment, which was not observed in FOXO3a knockdown cells (FIG. 3F). These results indicated that FOXO3a increased HtrA2 transcription through direct binding to the HtrA2 promoter. We next investigated whether HtrA2 plays an important role in EpAb2-6-induced cell death. In this experiment, we used shRNA to knock down HtrA2 expression in colon cancer cells. In HtrA2 knockdown cells, EpAb2-6 treatment induced fewer apoptotic cells (FIG. 3G) and less disruption of mitochondrial membrane potential (FIG. 3H) than shLuc (control knockdown) cells. Hence, FOXO3a-mediated HtrA2 transcription appears to be involved in EpAb2-6-induced apoptosis.

### Example 4 EpCAM Expression is Positively Correlated with PD-L1 Stabilization

EGFR activation was reportedly associated with increased PD-L1 expression in mouse models of EGFR-driven lung cancer and bronchial epithelial cells with mutant EGFR expression, suggesting that EGFR signaling is crucial for triggering immune escape. Further investigations then demonstrated that increased EGFR signaling upregulates PD-L1 expression by various mechanisms. Moreover, EpCAM plays a crucial role in EGFR activation. Based on these findings, we decided to investigate whether EpCAM regulates PD-L1 expression via EGFR activation. Since HCT116 cells expression of PD-L1 is extremely low, H441 cells with high expression of PD-L1 were substituted for HCT116 cells as an experimental model for further studies.

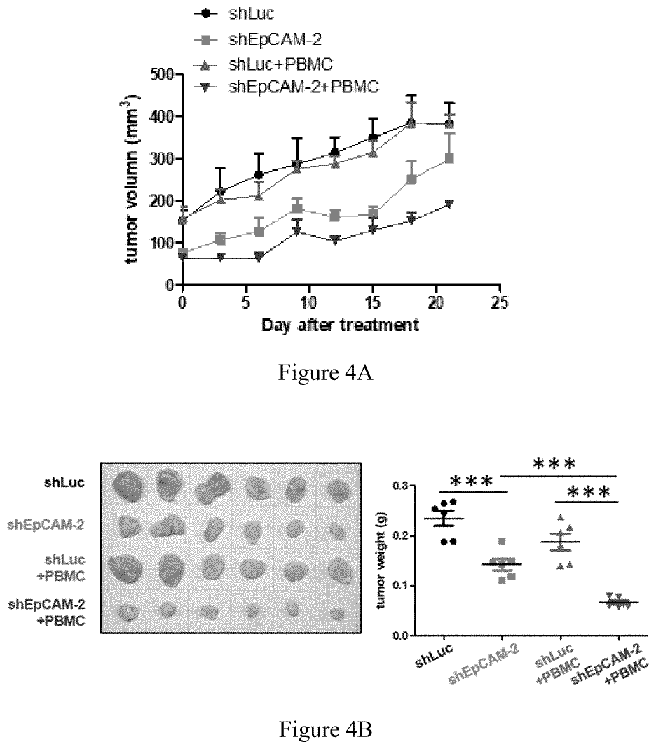

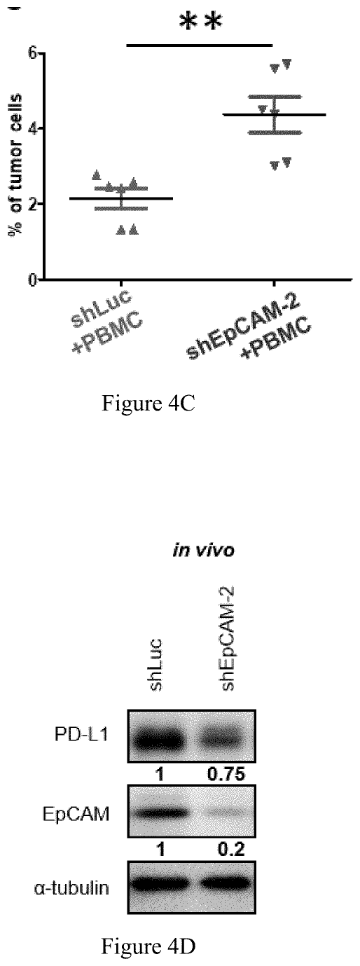

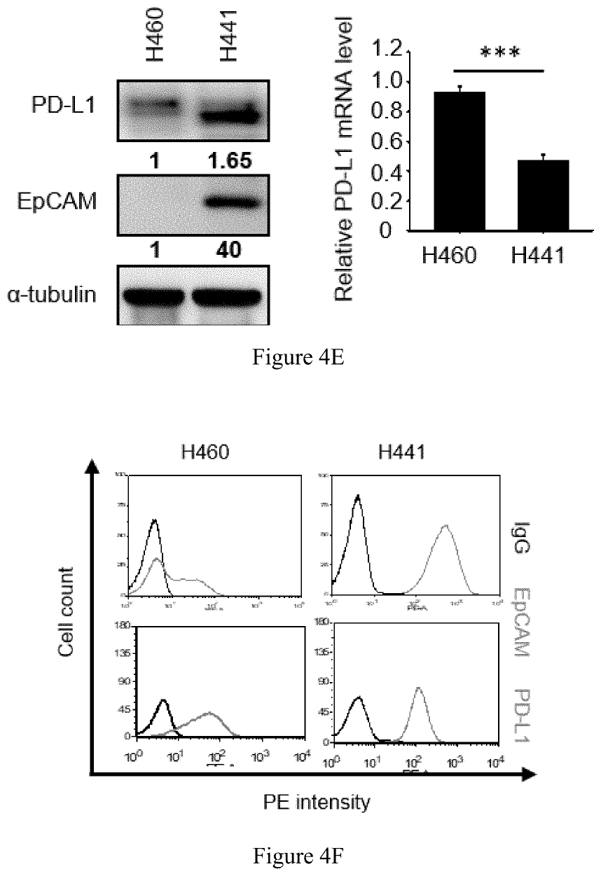

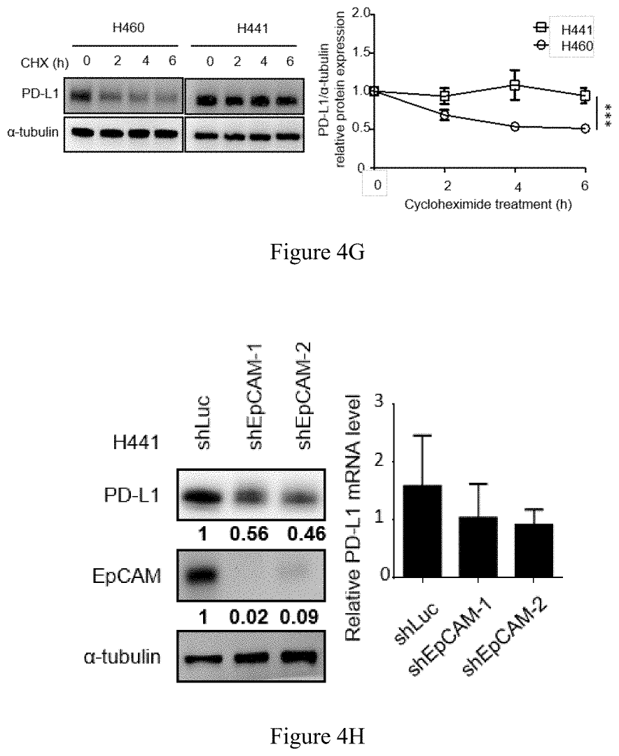

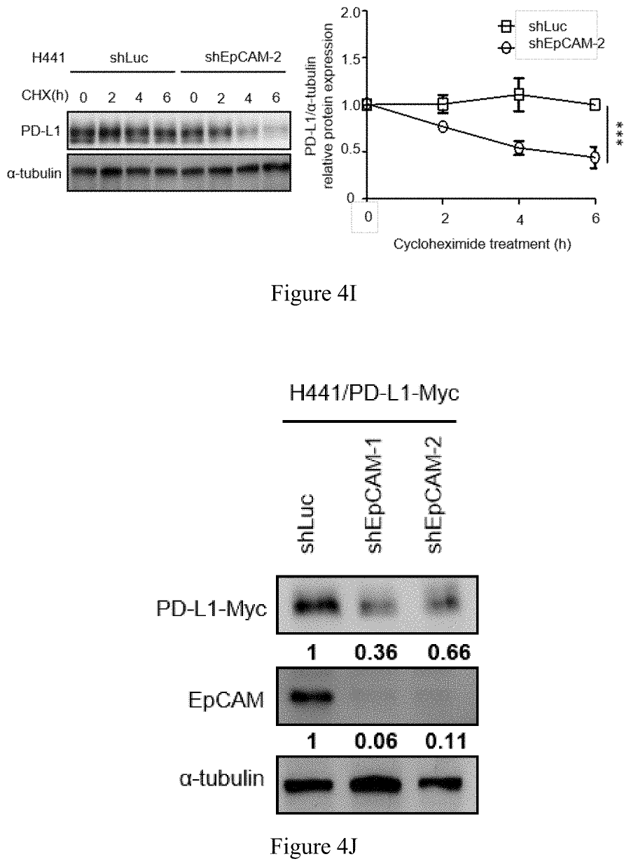

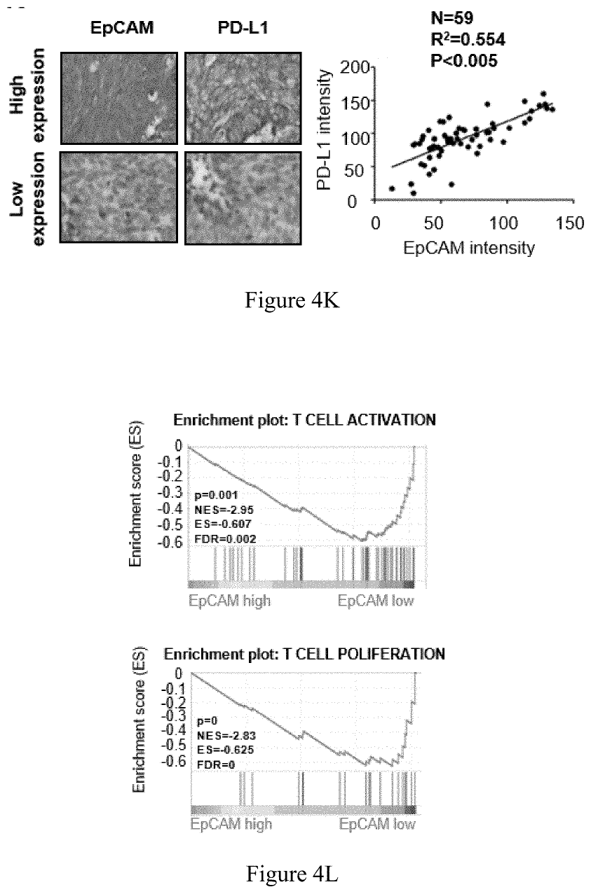

First, we wanted to clarify whether EpCAM is involved in escape from immune surveillance. Immunodeficient NSG mice carrying shLuc H441 xenografts in the left side and shEpCAM H441 xenografts in the right side were intravenously injected with PBMCs to reconstitute immune cells. EpCAM-knockdown cells exhibited decreased tumor weight, confirming that EpCAM promotes tumor progression (FIGS. 4A and 4B). Interestingly, shLuc tumors in mice with PBMC injection were not different from shLuc tumors in mice without PBMCs. However, shEpCAM tumors with PBMCs were smaller than those without PBMCs, implying that EpCAM might inhibit anti-tumor immunity (FIGS. 4A and 4B). By flow cytometry analysis, we found CD8⁺ T cells were enriched in shEpCAM tumor tissue (FIG. 4C), and Western blot analysis demonstrated that PD-L1 expression was diminished in shEpCAM tumor tissue (FIG. 4D). These results indicate that EpCAM promotes PD-L1 expression and reduces tumor-associated CD8⁺ T cells in vivo.

To investigate the correlation between EpCAM and PD-L1 expression, we analyzed PD-L1 mRNA expression in two NSCLC cell lines: H441 (high EpCAM expression) and H460 (no EpCAM expression). Compared to H441 cells, H460 cells exhibited a higher level of PD-L1 mRNA but a lower level of PD-L1 protein (FIG. 4E). Flow cytometry analysis of PD-L1 and EpCAM confirmed this finding (FIG. 4F). Stability of PD-L1 might serve as a key regulator of protein level. Indeed, a cycloheximide chase assay indicated that the half-life of PD-L1 in H460 cells was reduced compared to that in H441 cells (FIG. 4G). These findings suggest that the low PD-L1 protein level in H460 cells results from reduced stability of PD-L1 protein.

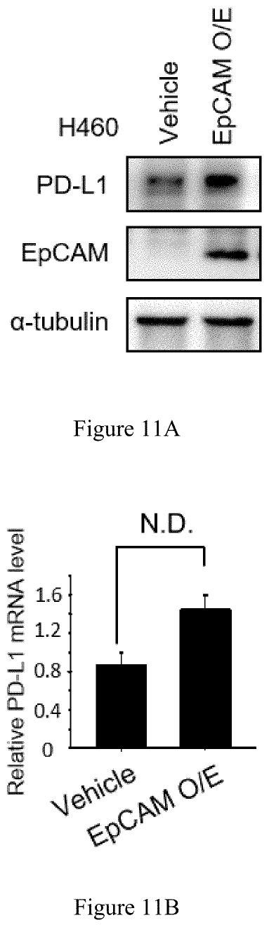

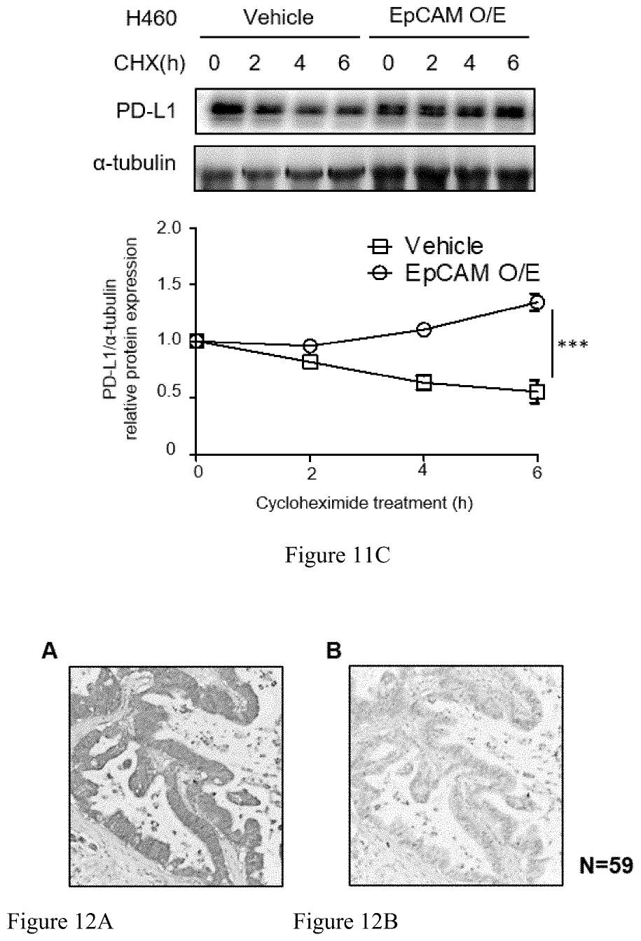

In order to investigate whether EpCAM influences PD-L1 protein stability, we overexpressed EpCAM in H460 cells (low endogenous PD-L1 expression and no EpCAM expression). We found EpCAM expression increased PD-L1 protein level but did not change its mRNA level (FIGS. 11A and 11B). A cycloheximide chase assay confirmed that PD-L1 protein half-life was increased by EpCAM overexpression (FIG. 11C). Additionally, knockdown of EpCAM in H441 cells (high endogenous EpCAM expression) decreased PD-L1 protein level and protein half-life but did not change mRNA level (FIGS. 4H and 4I). Consistent with this idea, H441 cells with CMV promoter-driven overexpression of PD-L1-Myc, which lacks endogenous regulatory elements, such as the PD-L1 promoter, 3′UTR and 5′UTR, also exhibited decreased PD-L1 protein when EpCAM was knocked down (FIG. 4J). Furthermore, to validate our findings in samples from human cancer patients, PD-L1 and EpCAM protein levels were examined in both lung and colon tumor specimens. Similar to our results in lung cancer cell lines, the PD-L1 protein level was highly correlated with EpCAM expression in a lung tumor tissue array (FIG. 4K). On the other hand, most of the specimens in the colon tissue array showed only weak staining for PD-L1, which prevented us from making a clear conclusion, even though the signal for EpCAM was strong (FIGS. 12A-12B). Furthermore, GSEA analysis indicated the related genes of T cells activation and proliferation enrichment in low EpCAM expression of lung cancer specimen (FIG. 4L). Accordingly, we concluded that EpCAM expression is positively correlated to PD-L1 protein stability.

### Example 5 EpEX Stabilizes PD-L1 Through the EGFR-ERK Pathway

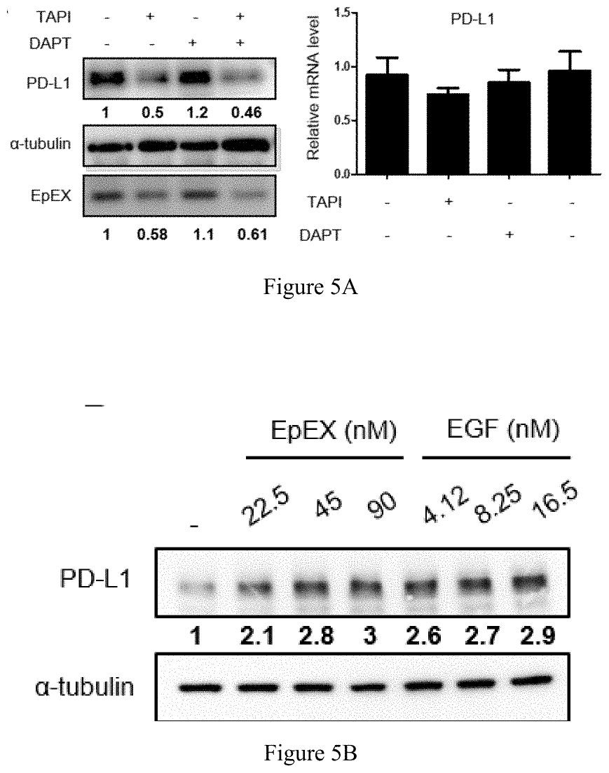

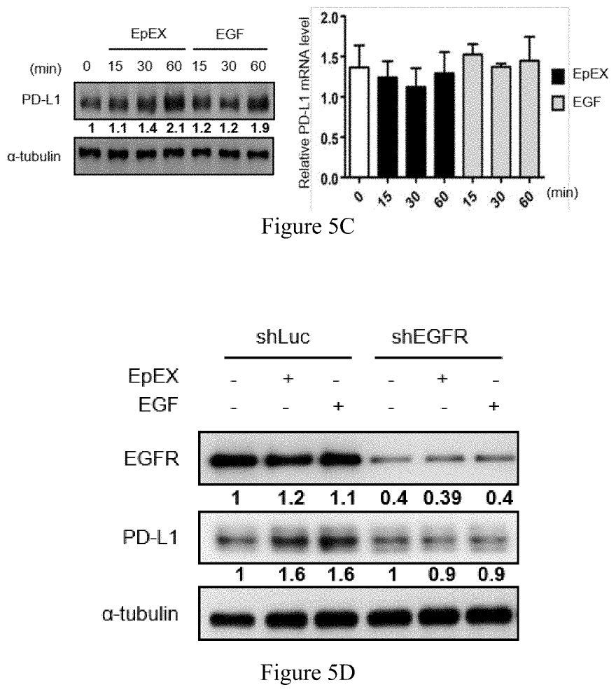

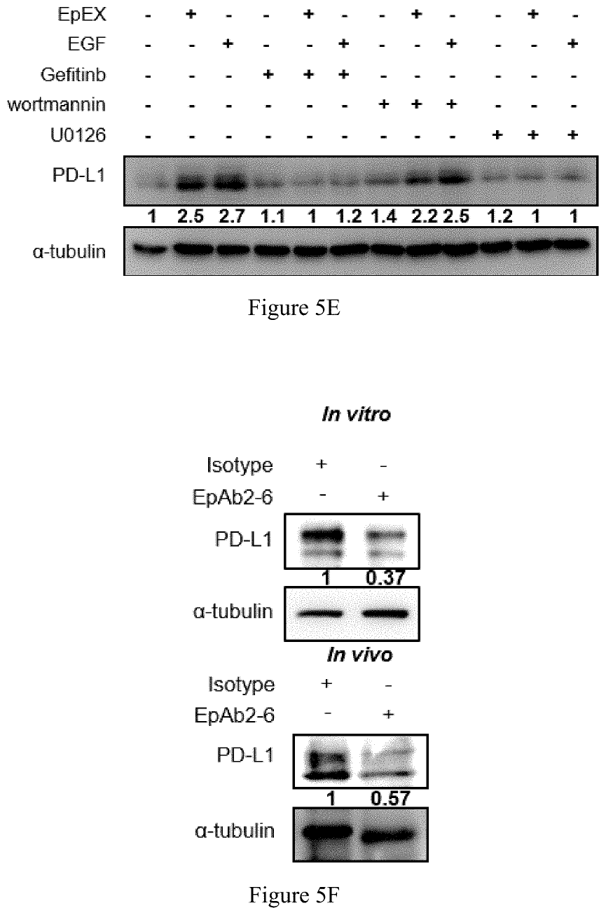

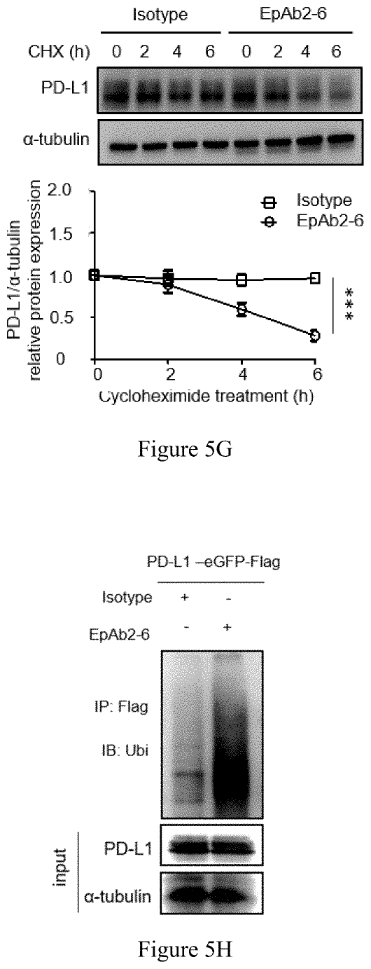

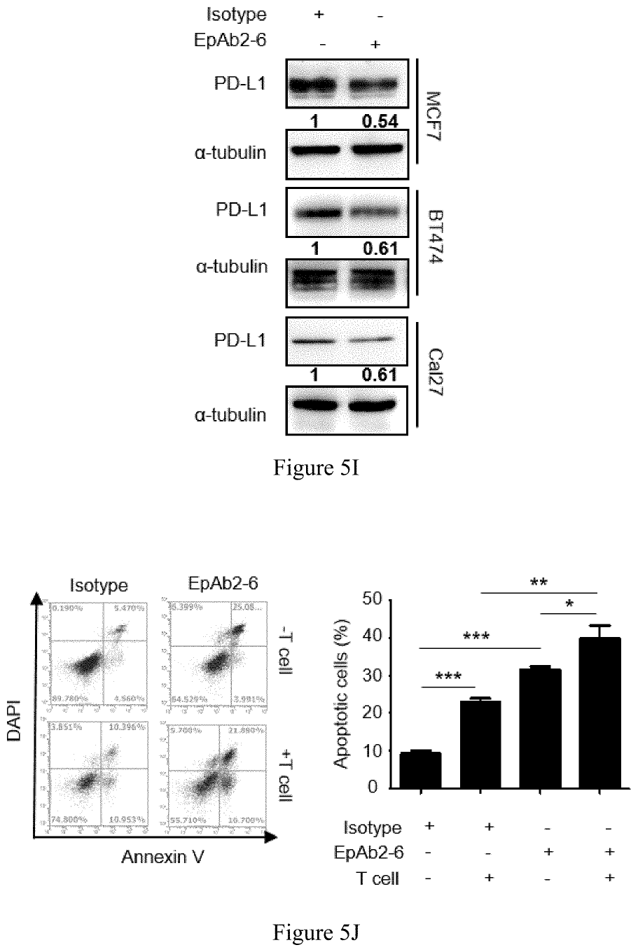

Activation of the EGFR signaling pathway can increase PD-L1 expression through various mechanisms. Thus, EpEX might regulate PD-L1 expression through EGFR signaling. Moreover, EpICD associates with FHL2 and β-catenin in a complex that translocates to the nucleus and transcribes downstream genes. Thus, we tested whether EpEX or EpICD is sufficient to cause PD-L1 stabilization. To test whether endogenous EpEX or EpICD are necessary for PD-L1 protein stabilization, ADAM17 inhibitor (TAPI) and γ-secretase inhibitor (DAPT) were utilized to prevent the generation of endogenous EpEX and EpICD, respectively. Blocking the shedding of endogenous EpEX, but not EpICD, caused a reduction PD-L1 protein level. Notably, no changes in PD-L1 mRNA levels were observed, suggesting that endogenous EpEX was crucial for PD-L1 protein stability (FIG. 5A). Moreover, our results showed that PD-L1 protein level was increased in a dose-dependent and time-dependent manner after H441 cells were treated with EpEX or EGF (FIGS. 5B and 5C). PD-L1 mRNA levels did not increase after EpEX or EGF treatment (FIG. 5C). These results suggest that EpEX can increase PD-L1 protein level, but EpICD cannot.

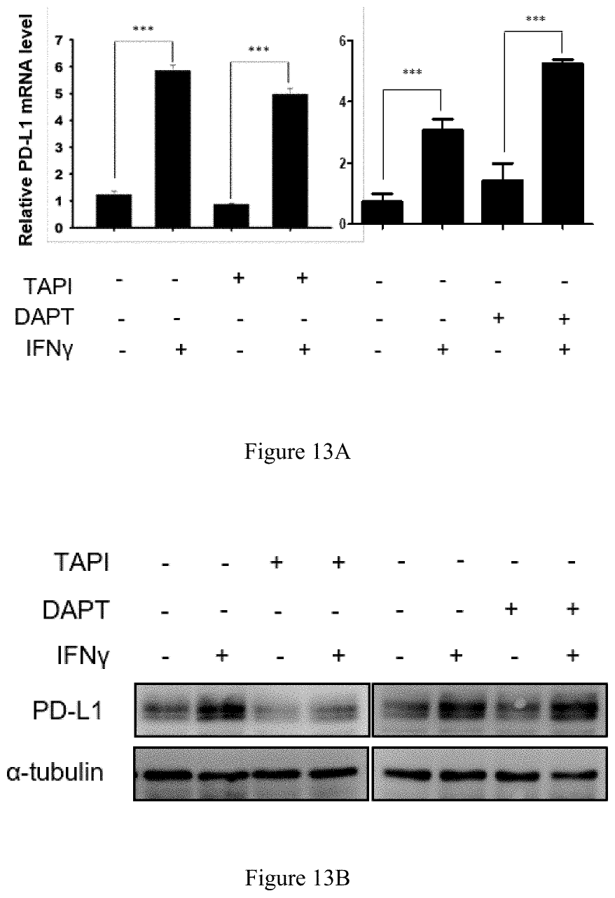

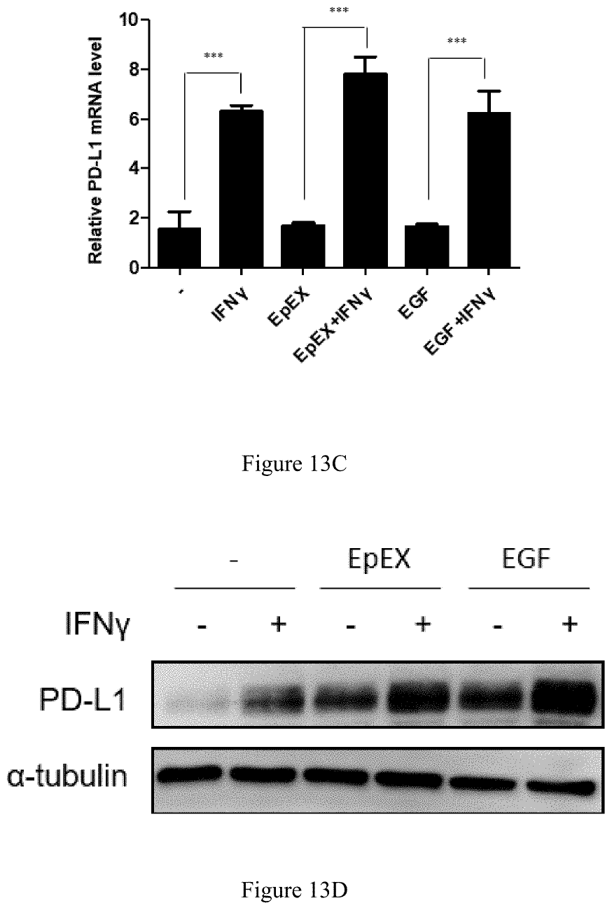

IFN-γ is known to induce PD-L1 expression through STAT family-mediated transcription. We next wondered whether endogenous EpEX and EpICD also participate in IFN-γ-mediated PD-L1 upregulation. PD-L1 mRNA was upregulated in H441 cells treated with IFN-γ& DMSO, IFN-γ & TAPI and IFN-γ & DAPT (FIG. 13A).

Surprisingly, the PD-L1 protein level was not increased as much in the presence of TAPI as it was in the IFN-γ & DMSO-treated cells (FIG. 13B). In other words, despite the fact that PD-L1 mRNA expression was elevated by IFN-γ stimulation, the protein level could not be fully upregulated without generation of EpEX. Additionally, PD-L1 protein expression was not impaired in IFN-γ & DAPT-treated cells (FIG. 13B). While PD-L1 mRNA level was increased in all cells receiving IFN-γ treatment, the protein level was only upregulated in those cells with IFN-γ & EpEX or IFN-γ & EGF treatment (FIGS. 13C and 13D). Thus, EpEX appears to be an essential factor for PD-L1 protein upregulation.

The EGFR signaling pathway is important for PD-L1 expression in cancer cells, and our previous study showed that EpEX can induce EGFR signaling. Therefore, we speculated that EpEX might act through EGFR signaling to prevent PD-L1 degradation. Indeed, shRNA knockdown of EGFR in H441 cells attenuated EpEX- or EGF-augmented PD-L1 levels (FIG. 5D). We next investigated which EGFR-downstream signaling pathway was involved in EpEX- and EGF-mediated upregulation of PD-L1. Treatment with Gefitinib abrogated both EpEX- and EGF-induced upregulation of PD-L1 protein level. Importantly, U0126, which is a MEK inhibitor but not an AKT inhibitor, abolished EpEX-mediated upregulation of PD-L1 as well (FIG. 5E). These results implied that, like EGF, EpEX-mediated upregulation of PD-L1 requires signaling through the MAPK pathway.

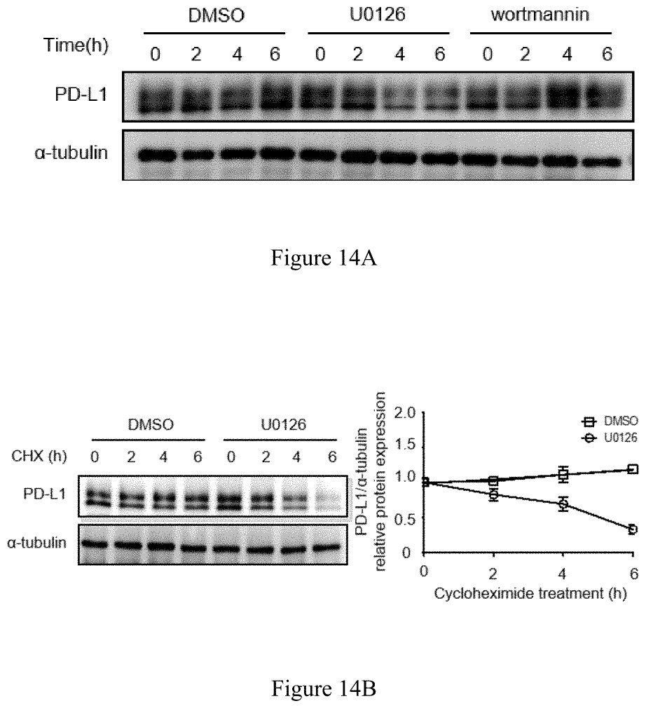

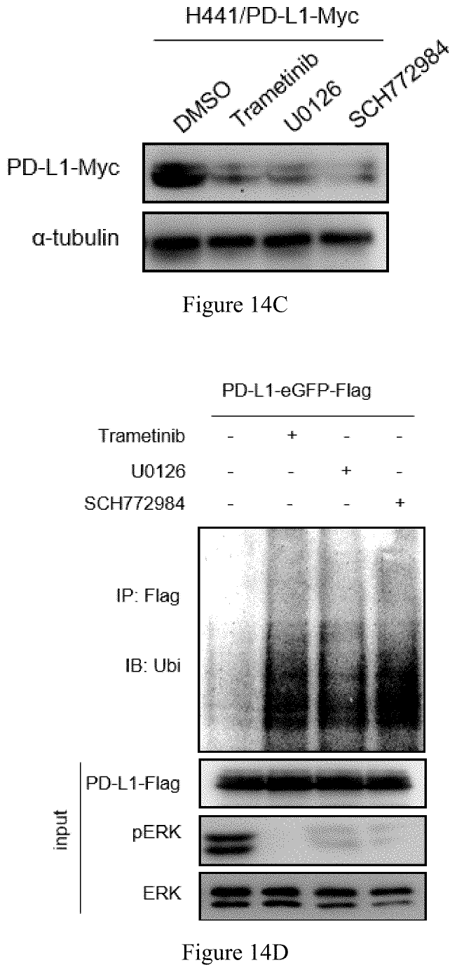

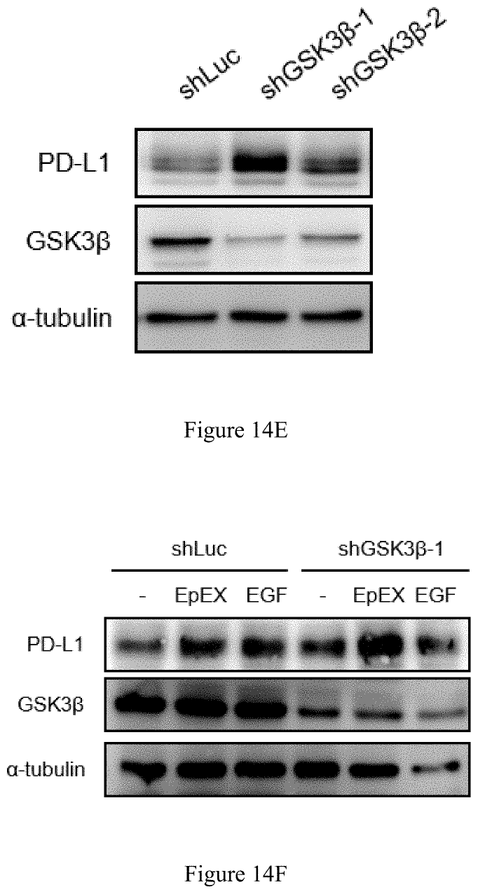

Both ERK and AKT signaling could regulate PD-L1 expression through different mechanisms. Hence, we wanted to know which signaling pathway was more important for PD-L1 expression in our system. Inhibition of ERK signaling but not AKT signaling decreased PD-L1 expression in a time-dependent manner (FIG. 14A). We then sought to verify that the MAPK pathway affects PD-L1 protein stability. In the presence of U0126 and cycloheximide, the turnover rate of PD-L1 was higher than it was in the DMSO control (FIG. 14B). Different inhibitors were then used to block the MAPK pathway in order to prevent misinterpretation of effects from nonspecific targeting. Indeed, the different MEK inhibitors, Trametinib and U0126, and ERK inhibitor, SCH772984, all decreased the PD-L1 protein level and increased polyubiquitination of PD-L1 (FIGS. 14C and 14D).

Glycosylation of PD-L1 at N192, N200 and N219 antagonizes GSK3β binding, which will normally induce phosphorylation-dependent proteasome degradation of PD-L1 by β-TrCP. As such, non-glycosylated PD-L1 is unstable compared to glycosylated PD-L1. Consistent with this mechanism, PD-L1 was upregulated in GSK3β-knockdown cells (FIG. 14E). However, EpEX and EGF could both induce PD-L1 upregulation in GSK3β-knockdown cells (FIG. 14F), suggesting that GSK3β is dispensable for EpEX- or EGF-mediated PD-L1 stabilization.

We next tested whether EpAb2-6 could attenuate PD-L1 upregulation and EpEX production. After EpAb2-6 treatment, PD-L1 protein level was downregulated both in vitro and in vivo (FIG. 5F). Consistent with our other findings, EpAb2-6 also decreased PD-L1 protein stability and increased polyubiquitination of PD-L1 (FIGS. 5G and 5H). Notably, EpAb2-6 treatment inhibited PD-L1 protein level in lung cancer cells and in cell lines derived from other EpCAM-positive cancer types, including breast cancer (BT474 and MCF7) and oral cancer (Ca127) (FIG. 5I). Using an apoptosis assay, we further found that co-culturing cancer cells with either EpAb2-6 or T cells could induce cancer cell apoptosis, and co-culturing cancer cells in the presence of both EpAb2-6 and T cells was more effective at inducing apoptosis (FIG. 5J).

### Example 6 EpAb2-6 Prolongs Survival in Metastatic and Orthotopic Mouse Models and Improves the Efficacy of Anti-PD-L1 Therapy in the PBMC-Cell Line-Derived Xenograft (CDX) Model

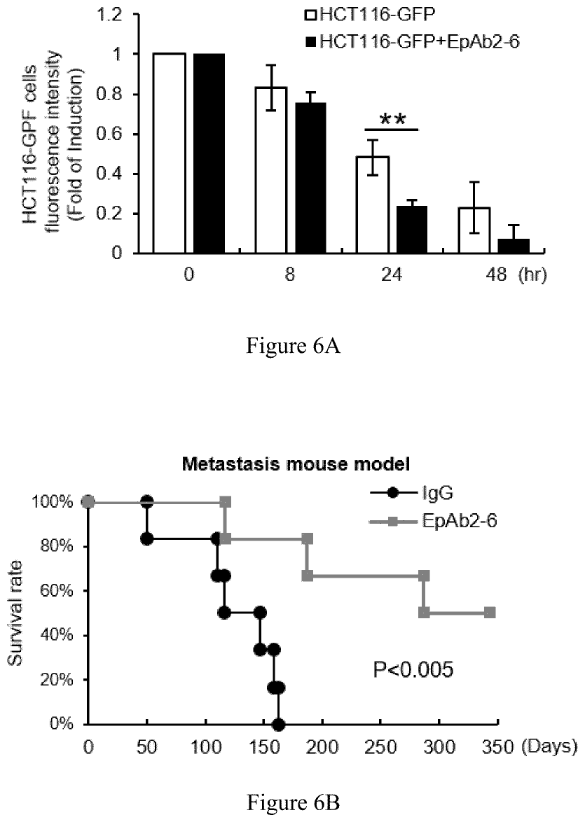

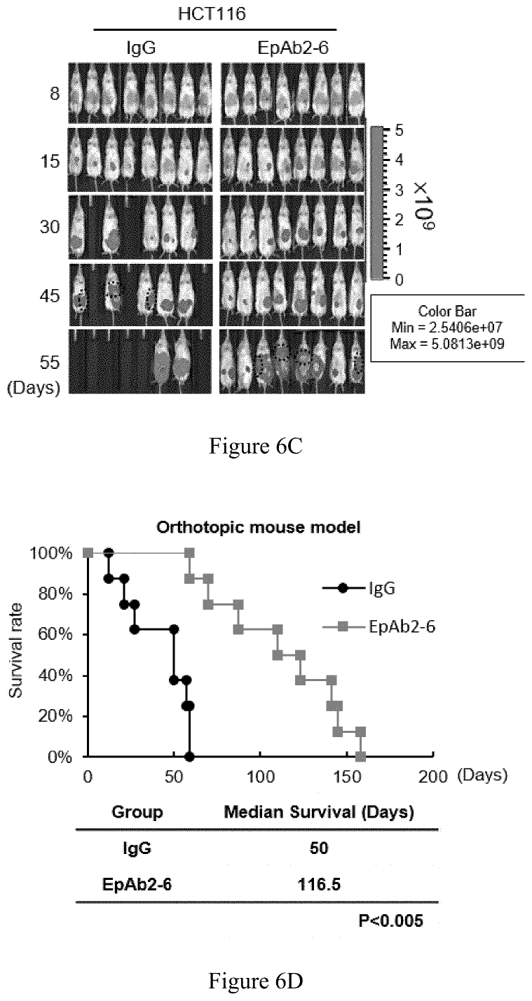

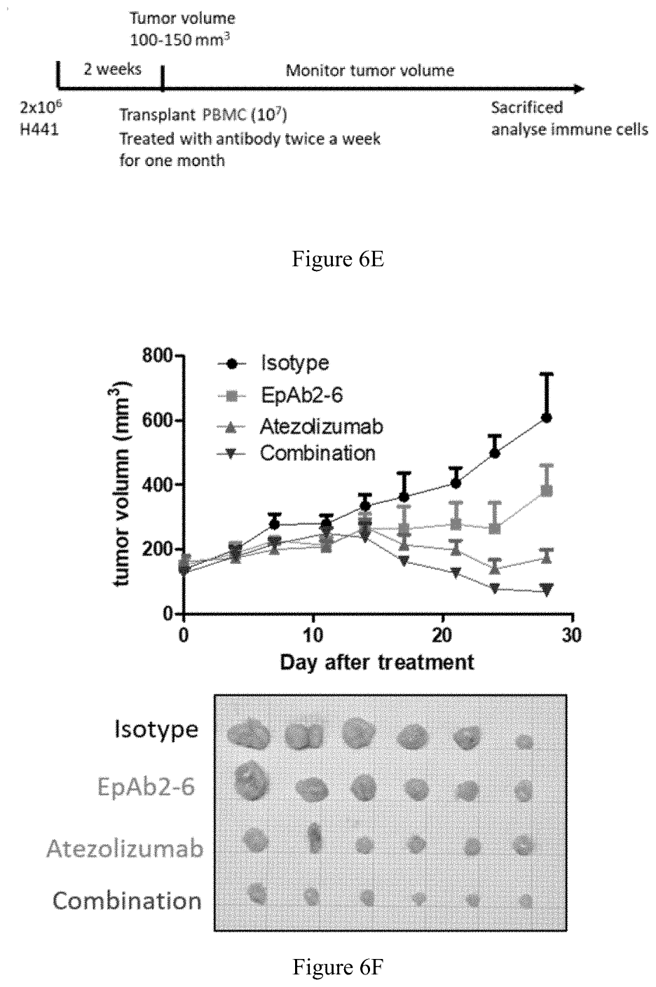

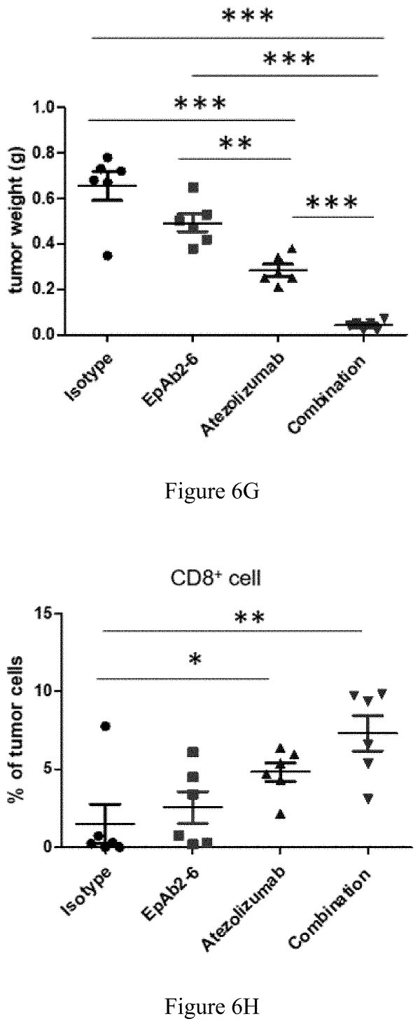

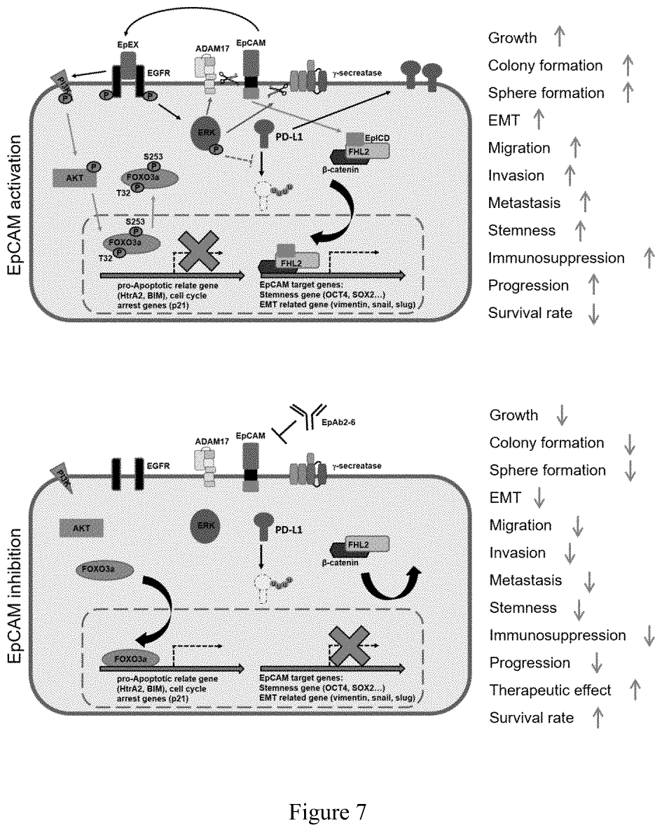

We next used an animal model of metastatic colon carcinoma to test whether EpAb2-6 treatment could increase the median overall survival of metastatic tumor-bearing mice. EpAb2-6 reduced the fluorescence intensity of mouse blood bearing circulating HCT116-GFP cells, suggesting that EpAb2-6 decreased the number of HCT116-GFP cells in mouse blood vessels in vivo (FIG. 6A). NOD/SCID mice were injected intravenously with SW620 cells and then intravenously treated with EpAb2-6, or an equivalent volume of control IgG, at 24 and 96 h after cell injection (antibody was delivered at 20 mg/kg/dose for a total dose of 40 mg/kg). The median survival time of the EpAb2-6 treatment group was significantly increased compared to the control IgG group (FIG. 6B, P<0.005 by the log-rank test).

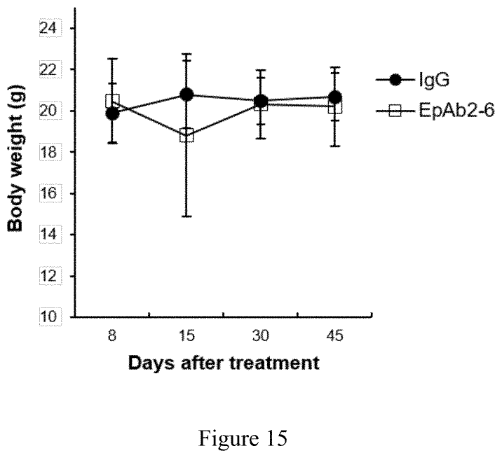

Because the subcutaneous model may not accurately reproduce human colon cancer biology, an orthotopic mouse model of colorectal cancer was also established to study the efficacy of our therapeutic antibody in the colorectal tumor microenvironment. We investigated the antitumor potential of EpAb2-6 in an orthotopic model with HCT116-Luc tumors that stably express firefly luciferase. Before the first therapeutic injection (8 days after tumor cell implantation), growing orthotopic tumors were monitored by bioluminescence imaging (FIG. 6C). Mice were treated with either control IgG or EpAb2-6 (20 mg/kg) every 2 days for 16 days. Notably, the EpAb2-6-treated group showed metastasis at 55 days, while the control group did so at 45 days (FIG. 6C). Moreover, no significant changes in body weight were observed during the treatment period (FIG. 15). At the end of the study, the median survival times for control IgG or EpAb2-6 treatment groups were 50 and 116.5 days, respectively (FIG. 6D). From these results, we conclude that EpAb2-6 treatment can prolong survival of tumor-bearing mice.

Based on our observations that EpAb2-6 treatment decreases PD-L1 expression in vitro and in vivo, we further wanted to evaluate whether EpAb2-6 could improve the therapeutic efficacy of anti-PD-L1 treatment in vivo. Because EpAb2-6 is not equipped with cross-reaction with mouse EpCAM, using syngeneic mouse model fails to evaluate the therapeutic efficacy of EpAb2-6 for immunotherapy. As illustrated in FIG. 6E, H441 cells were subcutaneously injected into NSG mice to establish the PBMC-H441-xenografted mice model. Two weeks later, mice were intravenously injected with 10⁷ PBMC cells and treated with Atezolizumab, an anti-PD-L1 antibody, and EpAb2-6 twice weekly for one month. Treatment with Atezolizumab or EpAb2-6 alone inhibited tumor growth in PBMC-H441 mice, and combination therapy produced a much better tumor inhibitory effect (FIGS. 6F and 6G). At the end of treatment, tumor tissues were collected, and CD8⁺ T cells were analyzed by flow cytometry. The CD8⁺ T cell population was increased in mice receiving combination therapy (FIG. 6H). Collectively, these data indicate that inhibition of EpCAM by EpAb2-6 may enhance the efficacy of anti-PD-L1 therapeutics in PBMC-H441-xenografted mice.

This is the first study to demonstrate that inhibition of EpCAM is correlated with an increase in HtrA2 gene expression. Moreover, this upregulation occurs through FOXO3a and induces apoptosis. EpEX increases PD-L1 protein stability and the combination immune therapy of anti-EpCAM and anti-PD-L1 antibodies provides a novel strategy for cancer therapy.

What is claimed is:

1. A method for treating, inhibiting or eliminating a cancer in a subject, comprising administering an effective amount of an inhibitor or an antagonist targeting to EGF-like domain I within the extracellular domain of EpCAM (EpEX) to the subject.

2. The method of claim 1, wherein the EGF-like domain I within the EpEX comprises a peptide consisting of amino acids 27 to 59 of EGF-like domain or a variant thereof that can bind to EGFR.

3. The method of claim 1, wherein the inhibitor or the antagonist targeting to EGF-like domain I within the EpEX is ribozymes, antisense oligonucleotides, short hairpin RNA (shRNA) molecules or small interfering RNA (siRNA) molecules that specifically inhibit and/or reduce the expression or activity of EpCAM.

4. The method of claim 1, wherein the inhibitor or the antagonist targeting to EGF-like domain I within the EpEX is a shRNA or a siRNA that knockdowns expression of EGFR, AKT, PD-L1 and/or MAPK and/or phosphorylation of FOXO3a and/or increases expression of HtrA2 and/or FOXO3a nuclear translocation.

5. The method of claim 4, wherein the shRNA comprises a nucleotide sequence consisting of GCAAATGGACACAAATTACAA (SEQ ID NO: 1) or a variant specifically inhibit and/or reduce the expression or activity of EpCAM.

6. The method of claim 1, wherein the inhibitor or the antagonist targeting to EGF-like domain I within the EpEX is a small molecule, peptide, an antibody or antibody fragment that can partially or completely block EpCAM activity.

7. The method of claim 1, wherein the inhibitor or the antagonist targeting to EGF-like domain I within the EpEX is an EpCAM-neutralizing antibody.

8. The method of claim 7, wherein the antibody is EpAb2-6 or a variant that can neutralize EpCAM.

9. The method of claim 1, which further comprises administering to the subject an inhibitor or an antagonist targeting to PD-L1 in an amount effective to inhibit expression or activation of PD-L1.

10. The method of claim 9, wherein the inhibitor or the antagonist targeting to PD-L1 is a PD-L1 checkpoint inhibitor.

11. The method of claim 10, wherein the PD-L1 checkpoint inhibitor is MEDI4736, atezolizumab, avelumab, or durvalumab.

12. The method of claim 9, wherein the inhibitor or the antagonist targeting to EGF-like domain I within the EpEX is intermittently, concurrently, separately or sequentially administered with the inhibitor or the antagonist targeting to PD-L1.

13. The method of claim 9, wherein the cancer is melanoma, renal cancer, prostate cancer, breast cancer, colorectal cancer, lung cancer, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer or T-cell lymphoma.

14. The method of claim 9, wherein the cancer is an EpCAM-overexpressing cancer, EGFR-overexpressing or activating cancer, AKT-overexpressing or overactivating cancer, MAPK-overexpressing or activating cancer, FOXO3a-inactivating cancer, HtrA2-inactivating cancer or PD-L1 expressing cancer.

15. The method of claim 9, wherein the cancer is a metastatic cancer or an advanced cancer.

16. The method of claim 9, wherein the cancer is a metastatic colorectal cancer or small cell lung cancer or an advanced colorectal cancer or small cell lung cancer.

17. The method of claim 9, wherein the subject is EpCAM-overexpressing.

18. The method of claim 9, wherein the subject who has been treated with at least one anti-cancer therapy or anti-cancer agent.

19. (canceled)

20. (canceled)

21. (canceled)

22. (canceled)

23. (canceled)

24. (canceled)

25. (canceled)
